(19) United States
(12) Patent Application Publication
(10) Pub. No.: US 2023/0252631 A1
(43) Pub. Date: Aug. 10, 2023

(54) NEURAL NETWORK APPARATUS FOR IDENTIFICATION, SEGMENTATION, AND TREATMENT OUTCOME PREDICTION FOR ANEURYSMS

(71) Applicant: MicroVention, Inc., Aliso Viejo, CA (US)

(72) Inventors: Satyananda Kashyap, San Jose, CA (US); Hakan Bulu, San Jose, CA (US); Ashutosh Jadhav, San Jose, CA (US); Ronak Dholakia, Aliso Viejo, CA (US); Amon Y. Liu, Redwood City, CA (US); Hussain S. Rangwala, Villa Park, CA (US); William R. Patterson, Huntington Beach, CA (US); Mehdi Moradi, San Jose, CA (US)

(21) Appl. No.: 18/165,912

(22) Filed: Feb. 7, 2023

Related U.S. Application Data
(60) Provisional application No. 63/307,467, filed on Feb. 7, 2022.

(51) Int. Cl. G06T 7/00; G16H 30/40; G16H 50/20; G06T 7/11

(52) U.S. Cl. CPC G06T 7/0012; G16H 30/40; G16H 50/20; G06T 7/11; G06T 2207/20084; G06T 2207/30101

(57) ABSTRACT

A neural network apparatus receives, as input from a user device, digital imaging information and the clinical information for an aneurysm patient and generates, using a neural network trained for aneurysm outcome prediction, the digital imaging information, and the clinical information, an outcome prediction for at least one intrasaccular implant device for implant in an aneurysm sac identified in the digital imaging information and having a highest predicted likelihood of complete occlusion of the aneurysm sac from a set of potential treatment devices. The apparatus is further configured to output, for display on a device, an identification of the at least one intrasaccular implant device and the outcome prediction for each of the at least one intrasaccular implant device.

### CROSS REFERENCE TO RELATED APPLICATION(S)

The present application for patent claims priority to Provisional Application No. 63/307,467 entitled “NEW DEEP NEURAL SEGMENTATION NETWORK FOR CEREBRAL ANEURYSMS IN 2D DIGITAL SUBTRACTION ANGIOGRAPHY” filed Feb. 7, 2022, assigned to the assignee hereof and hereby expressly incorporated by reference herein.

### TECHNICAL FIELD

The disclosure relates generally to the field of endovascular treatment, and more particularly, and not by way of limitation, some aspects relate to tools to assist in treatment of cerebral aneurysms.

### INTRODUCTION

An aneurysm is the abnormal focal dilatation of an artery, resulting from weakening of vessel walls with potential for rupture. The weak or thin spot on the artery in the brain balloons or bulges out and fills with blood, and a rupture of the aneurysm can have a mass effect on the nerves or brain tissue. Cerebral aneurysms often appear at arterial bifurcation points, particularly as wide-necked bifurcation aneurysms (WNBA), since locations with disturbed blood flow are more susceptible to aneurysm development. Different sources report rates of 1-5% of the population with asymptomatic intracranial aneurysms. If the aneurysm ruptures, the resulting subarachnoid hemorrhage can cause serious health problems such as hemorrhagic stroke, brain damage, coma, and death. The chances of successful treatment are higher when the aneurysm is discovered incidentally.

A major advance in endovascular treatment of cerebral aneurysms came with the introduction of Guglielmi Detachable Coils (GDC). GDC are bare-platinum coils that may be deployed within the aneurysm sac to promote thrombosis and aneurysm occlusion. Additional advancement in endovascular aneurysm treatment was provided through the development of flow diverting devices, such as intracranial flow-diverting stents (FDS). The first product of this type was approved by the US Food and Drug Administration (FDA) in 2011 (FDA Pre-Market Approval: P100018). Flow diverters include finely woven mesh stents that divert the blood flow from the aneurysm sac. Such devices may be designed to be placed in the lumen of the parent vessel at the point of bifurcation. Various innovative devices have changed the landscape of treatment options. One of these is the Woven EndoBridge (WEB) embolization system (MicroVention, A Terumo Group Company), approved by FDA in late 2018 (FDA Pre-Market Approval: P170032). The WEB device is an intrasaccular braided implant that is placed within the aneurysm sac. The WEB device may be used on ruptured and unruptured aneurysms.

The success of treatment with an intrasaccular device depends on complete occlusion of the blood flow into the aneurysm sac. As a result, the correct choice of the size of the intrasaccular device size for the given aneurysm is an extremely important step in treatment planning. Accordingly, a need exists for improvements in measurements of aneurysm sacs, as well as improvements in sizing for treatment devices.

### SUMMARY

In view of the above-described problems and unmet needs, the following presents a simplified summary of one or more aspects in order to provide a basic understanding of such aspects. This summary is not an extensive overview of all contemplated aspects. This summary does not identify key or critical elements of all aspects and does not delineate the scope of any or all aspects. The sole purpose of this summary is to present some concepts of the one or more aspects in a simplified form as a prelude to the more detailed description that is presented later.

In an aspect of the disclosure, a neural network apparatus, a method, and a computer-readable medium are provided for outcome predictions for intrasaccular implant devices based on angiography imaging and clinical information. The neural network apparatus receives, as input from a user device, digital imaging information and clinical information for the aneurysm patient and generates, using a neural network trained that is pre-trained for classification of objects, an outcome prediction for at least one intrasaccular implant device for implant in an aneurysm sac identified in the digital imaging information. The outcome prediction classified by the neural network based on a predicted likelihood for complete occlusion of the aneurysm sac based on the received digital imaging information and the clinical information. The neural network apparatus is further configured to output, for display at the user device in response to the input from the user device, an identification of the at least one intrasaccular implant device and the outcome prediction for each of the at least one intrasaccular implant device.

In another aspect, a system, an apparatus, a method, and a computer-readable medium are provided for outcome predictions for intrasaccular implant devices based on imaging and clinical information. In order to illustrate the concept, some examples are provided for angiography imaging. However, the concepts presented herein can also be employed for other types of digital imaging methods, such as non-angiography technologies, including, but not limited to, optical coherence tomography, positron tomography, near-infrared (IR) spectroscopy, and/or ultrasound based technology and imaging information. The system is configured to receive, as input from a user device, at least one of imaging information or the clinical information associated with an aneurysm patient and to generate an outcome prediction for an aneurysm treatment of the aneurysm patient based on the at least one of the imaging information or the clinical information received as input. The system is configured to send, to the user device, the outcome prediction for the aneurysm treatment for display at the user device.

In another aspect, a system, an apparatus, a method, and a computer-readable medium are provided for semi-automatic or automatic segmentation of an aneurysm sac. The apparatus is configured to receive, as input from a user device, digital information (e.g., angiography information, CT images, among other examples) for a patient and segment the aneurysm sac within the digital information using a trained neural network model with an encoder comprising a convolutional neural network that is pre-trained for classification of objects. The apparatus is configured to send, to the user device, segmentation information for the aneurysm sac.

The features and advantages described in the specification are not all-inclusive. These features are indicative of but a few of the various ways in which the principles of various aspects may be employed. Additional advantages and novel features of these aspects will be set forth in part in the description that follows, and in part will become more apparent to one of ordinary skill in the art in view of the drawings, specification, and claims and based on practice of the invention. Moreover, it should be noted that the language used in the specification has been principally selected for readability and instructional purposes and may not have been selected to delineate or circumscribe the disclosed subject matter.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of example aspects and implementations and, together with the description, further serve to explain the principles involved and to enable a person skilled in the relevant art(s) to make and use the disclosed technologies.

FIG. 1A is a diagram illustrating an 2D Digital subtraction angiography (DSA) image that may be used to measure the height, dome width and the neck size of the aneurysm sac, in both AP and lateral views in connection with aspects of the present disclosure.

FIG. 1B illustrates an example treatment device for an aneurysm, which may be considered in connection with aspects of the present disclosure.

FIG. 1C illustrates an example aneurysm sac having an implanted treatment device.

FIG. 2 illustrates an example system that includes an aneurysm treatment assistance service in connection with aspects of the present disclosure.

FIG. 3 illustrates an example aneurysm treatment assistance service in connection with aspects of the present disclosure.

FIG. 4A and FIG. 4B illustrate example user interfaces to receive user input and provide output to a user in connection with aspects of the present disclosure.

FIG. 4C illustrates an example predictive model building workflow in connection with aspects of the present disclosure.

FIG. 5 is a diagram illustrating a proposed architecture for segmentation network in connection with aspects of the present disclosure.

FIG. 6A and FIG. 6B illustrate examples of the performance for segmentation of detected aneurysm sac in connection with aspects of the present disclosure.

FIG. 7A illustrates an example 3D mesh model of an aneurysm sac reconstructed from segmented contours in connection with aspects of the present disclosure.

FIG. 7B illustrates examples of the performance for segmentation of detected aneurysm sac in connection with aspects of the present disclosure.

FIG. 8A and FIG. 8B are flowcharts showing a methods of operation of an aneurysm treatment assistance service in connection with aspects of the present disclosure.

FIG. 9A is a flowchart showing a method of operation of an aneurysm treatment module in connection with aspects of the present disclosure.

FIG. 9B is a flowchart showing a method of requesting and receiving aneurysm treatment assistance at a user device in connection with aspects of the present disclosure.

FIG. 10 illustrates a computer system that may implement the aspects of the aneurysm treatment assistance service in connection with aspects of the present disclosure.

The figures and the following description describe aspects of the present disclosure by way of illustration only. One skilled in the art will readily recognize from the following description that alternatives of the structures and methods illustrated herein may be employed without departing from the principles described herein. Reference will now be made in detail to several example implementations, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

The accurate measurement of the dimensions of an aneurysm sac is an important step in treatment planning for cerebral aneurysms. In some aspects, imaging information is taken in a pre-operative setting. Marking or delineating the contour of the aneurysm sac may be performed by an expert neuro-interventional radiologist and can be a time consuming and expensive aspect performed in a time critical situation of implant selection. Aspects presented herein provide tools with additional assistance information to assist in measurement and implant selection.

Aspects presented herein provide a semi-automatic, and a fully automatic method for segmenting the aneurysm sac based on imaging study information and/or clinical information input to a neural network or machine learning model. Aspects additionally provide for the automatic identification of a treatment device or a set of potential treatment devices to be implanted in the aneurysm sac. Aspects additionally provide for an automated prediction of outcome results associated with the identified treatment device or set of treatment devices. The present disclosure provides decision support software systems as a tool to assist in identifying a promising treatment at a rapid pace. The automated detection and/or segmentation, the identification of a potential treatment device (e.g., including a size or model), and/or the information about a predicted outcome of such treatment provides added tools for a user to consider in developing a treatment plan for an aneurysm. The automated aspects reduce the time for identification, segmentation, and identification of treatment devices, which can increase the pace at which treatment can be delivered to a patient. Aspects include the use of a neural network or neural network component that enables the additional consideration of a history of other data including prior aneurysm data in performing the detection, identification, and segmentation. The feedback aspects enable the model to continually improve the output provided to users. The combined use of imaging data and clinical information provides a more accurate and efficient estimation or prediction. The added efficiency and accuracy provide a tool that assists users in developing treatment plans.

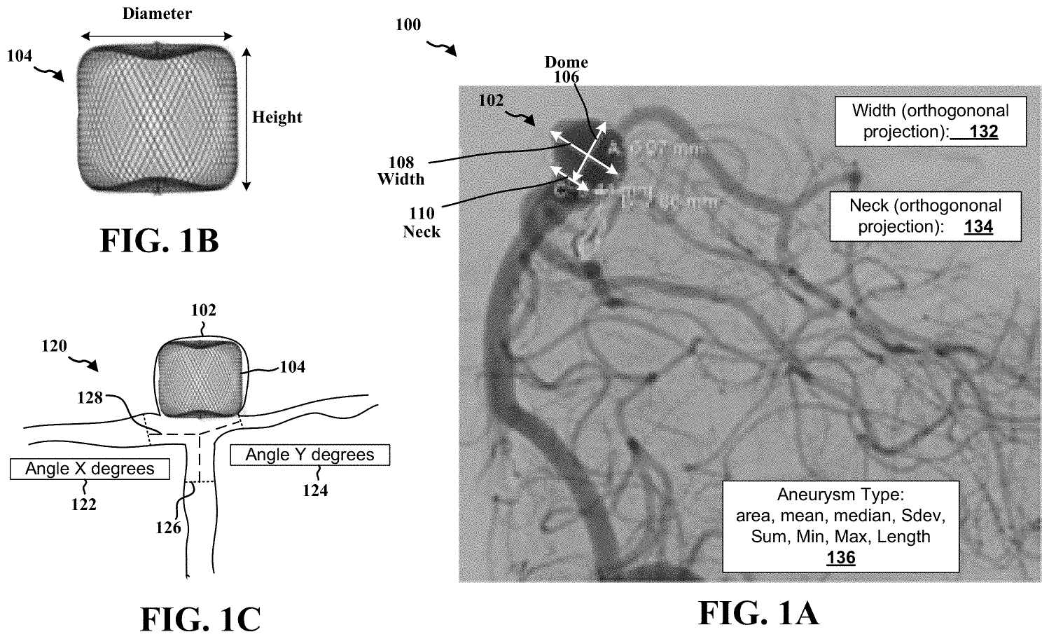

The success of treatment with an intrasaccular device, such as an intrasaccular embolization device, may depend on complete occlusion of the blood flow into the aneurysm sac. The completeness of the occlusion is affected by the size choice for a treatment device. A clinician, such as an interventional neuroradiologist (INR), may make an angiographic study of the vessel complex and measure key dimensions of the aneurysm sac. As an example, the clinician may perform a pre-operative digital subtraction angiography (DSA), intra-operative DSA, or other imaging systems such as, but not limited to, optical coherence tomography, positron tomography, near-IR spectroscopy, and ultrasound. The clinician may measure the height of the aneurysm sac, the dome width of the aneurysm sac, and the neck size of the aneurysm sac on both lateral and anterior-posterior (AP) views on DSA images. FIG. 1A illustrates an example image of an aneurysm sac 102, and FIG. 1B illustrates an example treatment device 104 that may be implanted at the aneurysm sac. In FIG. 1A, the image is a 2D Digital subtraction angiography (DSA) image 100 that may be used to measure the height 106, dome width 108, and the neck size 110 of the aneurysm sac 102. As an example, the clinician may perform the measurements manually, and may perform the measurement in multiple views such as in both AP and lateral views. The treatment device 104 in FIG. 1B is a woven embolization type device, and is one of various types of treatment devices that can be considered in connection with the present disclosure. As well, a particular type of treatment device may include various models, each having a maximum height and diameter in the implant model's fully open state. FIG. 1B illustrates a height and diameter dimension for the treatment device 104. The size of the treatment device may be selected based on these measurements to ensure correct fit and device activation. The size of the treatment device 104 in FIG. 1B is not proportional to the DSA image in FIG. 1A.

FIG. 1C illustrates an example diagram 120 showing the treatment device 104 implanted within an aneurysm sac 102 to disrupt the flow into the aneurysm sac. In some aspects, the treatment device may come in various combinations of height and diameter. Table 1 illustrates an example set of different diameter and height measurements for various example models. Table 1 only presents a few example dimensions in order to illustrate the concept, and various additional combinations of diameter and height may be considered for selection of a treatment device for a particular aneurysm treatment.

TABLE 1

| Device Model | Diameter | Height |
|---|---|---|
| A | 4 | 2 |
| B | 4 | 3 |
| C | 4.5 | 2 |
| D | 4.5 | 3 |
| E | 5 | 2 |
| F | 6 | 3 |
| G | 6 | 4 |
| H | 7 | 3 |
| I | 7 | 4 |
| J | 7 | 5 |
| K | 8 | 3 |
| . . . | . . . | . . . |

In some aspects, the largest two dimensions of the aneurysm sac 102 may be used to determine the diameter/height combination of the treatment device 104 to be implanted into the aneurysm sac 102. DSA may be used for measuring the dimensions and characteristics of aneurysms as a step in planning interventional treatments. Incorrect sizing of the aneurysm sac may lead to incomplete treatment by leading to use of an intrasaccular implant device that is too small or too large to provide effective treatment of the aneurysm. An intrasaccular flow disruptor, such as the treatment device shown in FIG. 1B and FIG. 1C, treats aneurysms by diverting the blood flow from the aneurysm sac. Residual blood flow into the aneurysm sac after intervention by implanting the treatment device 104

To avoid under-sizing the treatment device 104 to be implanted, the aneurysm sac dimensions may be measured on the working projection DSA image, which may show the maximum dimensions of the sac prior to the procedure. Aspects presented herein provide an automatic, or semi-automatic, reliable, and fast algorithm to segment the aneurysm sac and/or to identify potential treatment devices with predicted treatment outcome, which may decrease the time of measurement and improve the accuracy of implant selection. For example, aspects presented herein may help to improve the selection of the treatment device 104 in order to minimize residual blood flow into the aneurysm sac after the treatment device 104 is implanted. Aspects presented herein further provide a tool based on transfer learning to train a highly accurate segmentation deep neural network for contouring the sac of wide-necked bifurcation aneurysms in a more efficient and reliable manner, identifying a device for implant, and providing a prediction of outcome associated with the identified device based on imaging data and/or clinical information.

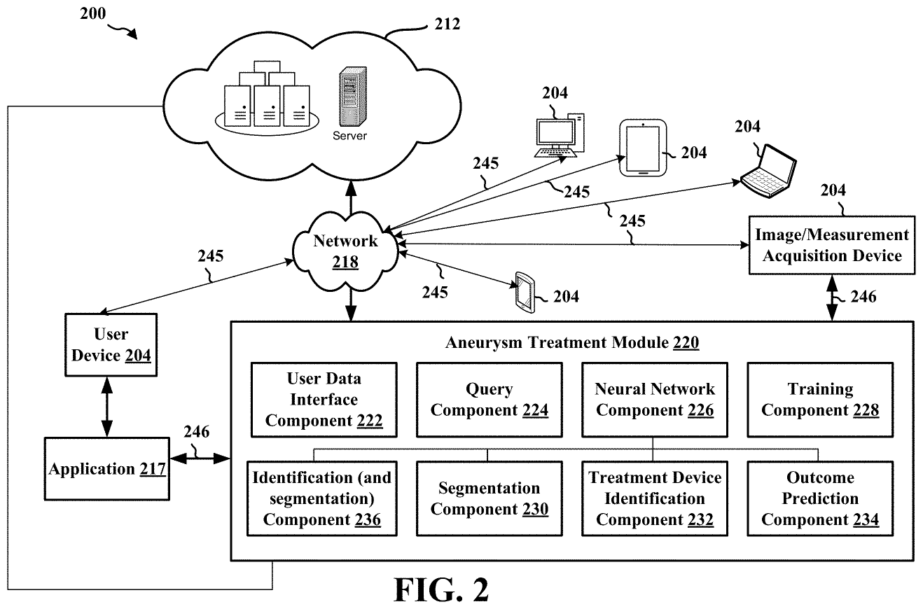

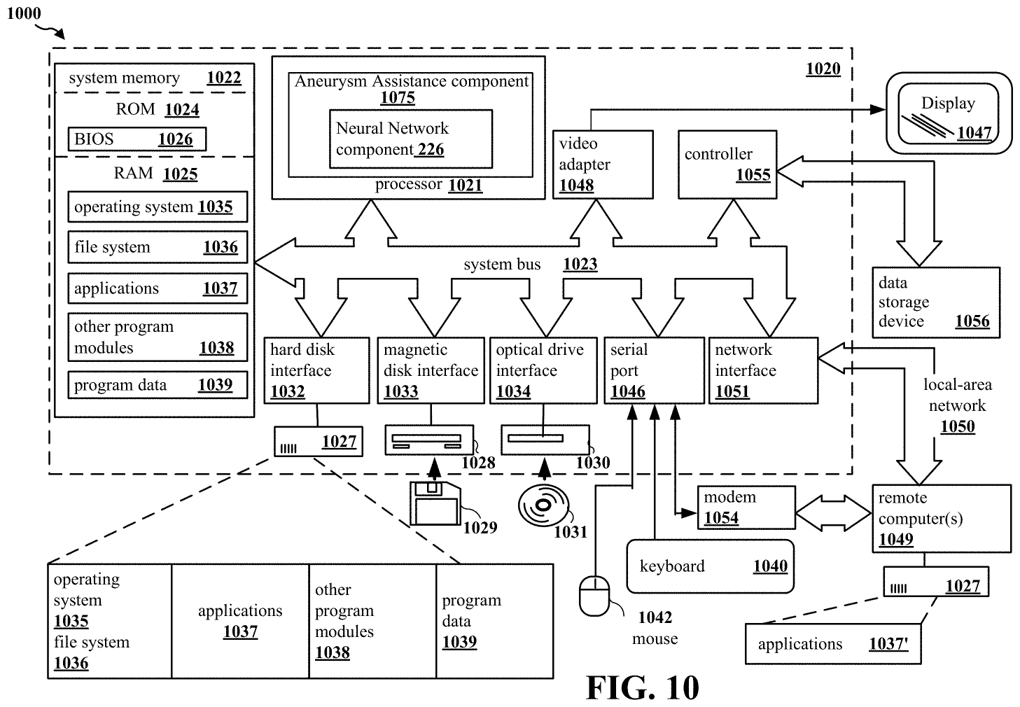

FIG. 2 illustrates a diagram showing an example system 200 that provides a tool that aids in planning treatment for aneurysms including the selection of a treatment device using an automatic, or semi-automatic, reliable, and efficient algorithm to segment the aneurysm sac and/or to identify potential treatment devices with predicted treatment outcome. FIG. 2 illustrates that an aneurysm treatment module 220 (which may also be referred to as an aneurysm treatment planning tool) that is configured to receive patient imaging study information and/or clinical information from one or more user devices 204 and, in response, to provide automated measurements and/or predictive information. The reception of the imaging study information and/or clinical information may be received by the module 220 via a user data interface component 222, which may be a communication interface that enables data to be transferred between one or more user devise 204 and the aneurysm treatment module 220. FIG. 10 illustrates a computer system 1000 that may implement the aspects of the aneurysm treatment module, and shows various examples of communication interfaces that may be used to receive the imaging study information and/or clinical information for a patient.

FIG. 2 further illustrates a communication system usable in accordance with the present invention. The communication system includes one or more accessors (also referred to interchangeably herein as one or more “users”) that access the communication system via one or more user devices 204, which may also be referred to as terminals. In one aspect, data for use in accordance aspects presented herein, for example, input and/or accessed by the users via the user devices 204, such as personal computers (PCs), minicomputers, mainframe computers, microcomputers, servers, telephonic devices, tablets, or wireless devices, such as personal digital assistants (“PDAs”), a hand-held wireless devices, or an image or measurement acquisition device. The user devices 204 may be coupled to a server 212, such as a PC, minicomputer, mainframe computer, microcomputer, or other device having a processor and a repository for data and/or connection to a repository for data, via, for example, a network 218, such as the Internet or an intranet, and couplings 245. The couplings 245 may include, for example, wired, wireless, or fiberoptic links. In some aspects, the user device 204 may have a communication interface 246, connection, or communication link to the aneurysm treatment module 220, e.g., via an application 217. In some aspects, the method and system presented herein operate in a stand-alone environment, such as on a single terminal. For example, in some aspects, the aneurysm treatment module 220 may be a component within a user device 204 or as part of an image acquisition device or a measurement device, as an example user device 204, that may be used by the clinician to obtain image information or measurement information on a patient.

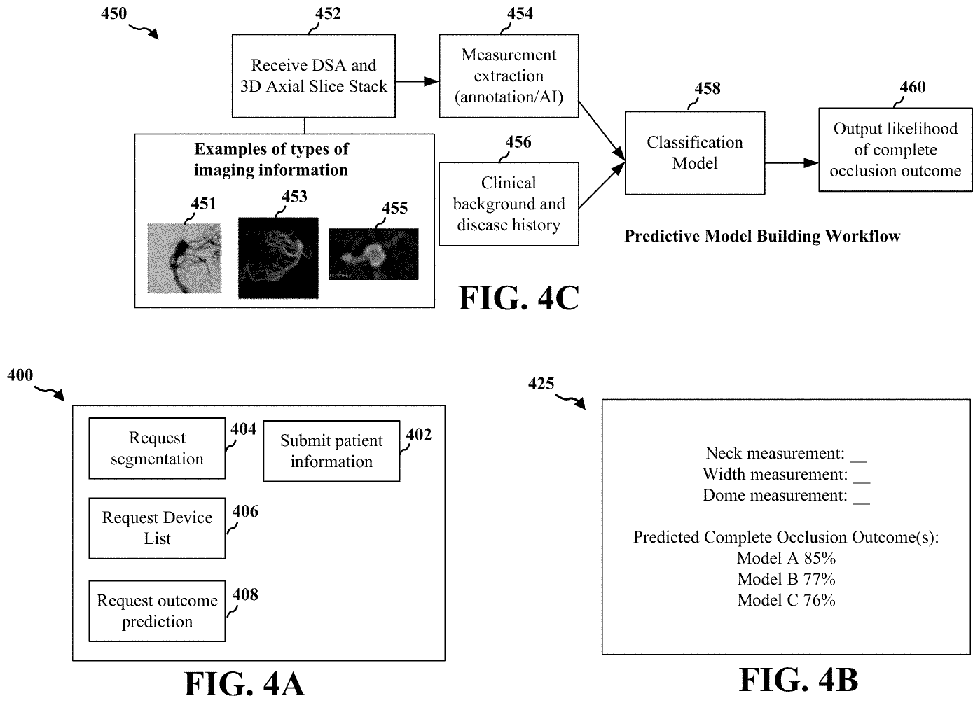

The module 220 may include a query component 224 configured to receive a user query for a measurement and/or predictive result. In some aspects, the automatic measurement or predictive information may be provided to the user device in response to receipt of the patient imaging information and/or clinical information, e.g., without a further query. In other aspects, the module 220 may be configured to return a measurement or predictive information to the user device in response to receipt of a particular user query. FIG. 4A illustrates an example user interface 400 that may be presented at a user device to enable the user device to submit, or enter, imaging study or clinical information for a patient, e.g., through submission component 402, and to request measurement or predictive information results from the module 220. As an example, the user device 204 may send a request, or query, for an automated, or semi-automated, measurement based on imaging study information, e.g., based on a user selection of a segmentation request option 404 presented to the user at the user interface 400. The request may be received via the query component 224. In response to the request, the module 220 may perform the automated measurement and send the measurement information to the user device 204. As another example, a user device 204 may send a request to the module 220 for an identification of a treatment device and/or a predicted outcome result for treatment with the treatment device, e.g., based on a user selection of a device identification request option 406 or an outcome prediction request option 408 presented to the user at the user interface 400. In response to the request, the module 220 may perform the automated measurement and send an identification of one or more potential treatment devices (e.g., type and/or size), with or without predicted treatment outcome information, to the user device 204. FIG. 4B illustrates an example user interface that shows an example of measurement and predictive information that may be provided, e.g., displayed, at a user interface 425 at a user device 204 based on the information generated at and sent from the module 220.

The module 220 may reside remotely on one or more servers connected to network 218. For example, module 220 may reside on server 212 and/or other servers on network 218, and the user devices 204 may send information and/or requests to the module 220 and receive output from the module 220 via the network 218. In other aspects, the module 220 may be comprised within or coupled directly to a user device 204.

The module 220 may include a neural network component 226, which may also be referred to as a neural network component, and artificial intelligence component, etc. The neural network component 226 uses a predictive model to provide the measurement information, treatment device identification, and/or the predicted treatment outcome for a patient, based on the received clinical background and imaging study information for that patient, e.g., that may be received via the user data interface component 222. Table 2 illustrates various examples of clinical information that may be received as user input.

TABLE 2

| No | Category | Features |
|---|---|---|
| 1 | Demographic | Age, gender, height, weight, race |
| 2 | Aneurysm | Location (anterior communicating artery complex, |
| | Information | Basilarex, Internal carotid artery terminus, Middle |
| | | cerebral artery bifurcation), side (right, left, |
| | | midline), type (ruptured, unruptured), the unruptured |
| | | aneurysm was detected by (incidentally, |
| | | symptomatically), Hunt and Hess grade, NIHSS |
| | | Score, mRS score |
| 3 | Dimensions | AP and Lateral view (height, width, neck) |
| | of Aneurysms | |
| 4 | Allergies | Known allergies and medication information |
| | and Drugs | |
| 5 | Pre-existing | Smoking history, substance abuse, affected body |
| | conditions | systems (neurological, psychological/psychiatric, |
| | | cardiovascular and circulatory, endocrine, |
| | | metabolic, musculoskeletal, eyes/ears/nose/throat/ |
| | | head/neck, respiratory, gastrointestinal, |
| | | genitourinary, hematological/lymphatic, |
| | | dermatological |

Pre-existing conditions may include information about a patient's existing clinical condition, which may be grouped into groups based on different body systems, as shown in the example in Table 2, or may be grouped or input in a different manner. As well, the clinical information relating to pre-existing conditions may be further grouped or input based on the grouping. As an example, under the cardiovascular and circulatory grouping, the possible health conditions may further include hypertensions, coronary artery disease, valve disease/dysfunction, hypotension, arrhythmia, myocardial infarction, angina, or heart failure, among other examples.

The measurement information may include various measurements from one or more views. As an example, the measurement information that is input may include a height, width, and/or dome measurement in an AP and lateral view from DSA images. The imaging data may include one or more views from any of various types of imaging sources. As an example, imaging data may be input based on magnetic resonance imaging (MRI), magnetic resonance angiography (MRA), or a computed tomography (CT) scan, among other examples. As presented herein, the cerebral arteriography information may be used to confirm the presence of an aneurysm and to evaluate which treatment options may be best suited for particular aneurysms based on size, shape and location. In some aspects, the imaging data may include two-dimensional (2D) DSA images. The images may be pre-operative imaging information. The images may be obtained within an operating suite prior to implanting an intrasaccular device for treatment of aneurysm, for example. In some aspects, the imaging data may include three-dimensional (3D) data, such as an axial slice stack reconstructed from a sequence of 3D-DSA images produced by a rotational C-arm angiography. Other examples of imaging include slice stacks (axial, sagittal, or coronal) contrast enhanced magnetic resonance angiography (MRA), computed tomography angiography (CTA) (such as DynaCTA or VasoCTA). The model presented herein may be trained to use any type of image information, e.g., 2D images, an axial slice stack reconstructed from a sequence of 3D-DSA images, among other examples. In some aspects, the imaging data information may be raw imaging information, e.g., that has not been marked, cropped, or annotated by a user to guide the segmentation. In other aspects, a user may input some additional input on the imaging information. For example, a user may select a rectangular area to be analyzed. The user may mark, crop, annotate, or otherwise adjust the imaging data prior to the imaging data being received in the system.

As an example of annotations that may be included as input either on imaging data input for a particular patient or as part of training data to train the neural network model, annotations may be included on 2D or 3D images to indicate dimensions and/or contour of the aneurysm sac. For example, an annotation may be added to image data, such as shown in FIG. 1A to indicate a height, a width, and/or a dome length of an aneurysm region in imaging information. As a further example, the annotations may be indicated on multiple orthogonal DSA projections, such as AP and lateral projections, to deliver multiple dimensions (e.g., six dimensions for two projections) for analysis. As an additional measurement, the area of the aneurysm sac may be included as an annotation. The area may be calculated, e.g., by a clinician marking a contour of the aneurysm. Similar to input for a particular patient, such measurements may be included with data sets of imaging information for training data to train the neural network component to perform automatic segmentation/measurement and/or automatic identification/detection of an aneurysm sac.

In some aspects, annotation information may include an annotation indicating a vessel geometry, size, and/or angle of bifurcation. As an example, the annotation may indicate diameters of the daughter branches and of a parent vessel. The annotation may indicate angles between each daughter vessel (e.g., a dominant and non-dominant) and the parent vessel. The annotations may include or be based on markings inserted by a clinician on images. FIG. 1C illustrates an example of markings 126 and 128 and angle information 122 and 124 that may be inserted as annotations with an image by a clinician. FIG. 1A illustrates an example of measurement information 132, 134, and 136 that may be inserted as annotations on by a clinician with an image. As an example, the angle measurements may include an angle between a parent vessel and a left daughter, e.g., at 122, and an angle between a parent vessel and a right daughter, e.g., at 124, as well as normalized variants of the angles. The normalized variants of the angles may correspond to vessel-normalized left angle=(vessel−left angle)/180. Diameters of the parent vessel, as shown at 126, as well as diameters of the left daughter branch, right daughter branch may be marked or measured. A larger daughter branch may be indicated, and a ratio of such measurements may be included as annotations.

In some aspects, as noted above, the imaging data may include 3D imaging data, such as a stack of reconstructed axial slices from an imaging study. The system may receive annotations such as a clinician entry indicating the contour of the perimeter of the aneurysm sac in the images in the stack where it is visible. The annotations may be used to reconstruct a volumetric mesh model of the aneurysm sac and to estimate the volume and surface area of the sac.

In some aspects, one or more lateral view measurements may be omitted in connection with imaging data, whether as input for a particular patient or as training data. In such aspects, aggregated measurements may be calculated from an AP and later view. As an example, for 3D features, the surface area and the volume of the aneurysm sac may be calculated from a 3D mesh model. A non-sphericity index (NSI) and/or isoperimetric ratio (IPR) may be calculated from the 3D mesh model.

Manual annotation of images for the purpose of calculating the imaging features may not be available or may take additional time to perform. Aspects presented herein provide a tool that enables and improves automating a calculation of measurements, e.g., 2D measurements and/or 3D measurements of area and volume, based on imaging data input to the service. The automated measurements may be performed efficiently and accurately to reduce the time to treatment. In some aspects, a step of delineating an aneurysm boundary, e.g., which may be performed by an expert neuro-interventional radiologist. The delineation step can be time consuming and expensive, which is performed in a time critical situation of implant selection. The tools presented herein can help to save time for the doctors and provide additional assistance information to assist them in measurement and implant selection.

For example, in some aspects, the neural network component 226 may include a segmentation component 230 that is configured to output semi-automated measurement information for an aneurysm sac based on imaging study information input for the patient. In some aspects, the neural network component 226 may include an identification component 236 that is configured to output automated, or semi-automated, identification information for an aneurysm sac based on imaging study information input for the patient. The neural network component 226 may include an identification component 236 configured to provide detection and segmentation of an aneurysm, whereas the segmentation component 230 may perform segmentation on an image with some user input, such as cropping, marking, annotating, or otherwise indicating the portion of the image for segmentation of the aneurysm. The detection or identification of the aneurysm in combination with the measurement or segmentation may be referred to as automated measurement. As presented herein, neural network or machine learning algorithms may be trained may be configured to automatically segment an aneurysm region from imaging data and use the measurements as features in a predictive modelling feature. In some aspects, deep learning based algorithms may be employed at the neural network component 226 as part of the segmentation component 230 and/or the identification component 236. In some aspects, different deep learning based algorithms may be provided for 2D imaging information and 3D imaging information.

### Segmentation

The process of marking the aneurysm sac in a DSA image may include first locating the sac in the image, and then carefully marking the sacs contour. Clinicians may complete the first step intuitively and quickly as they have prior knowledge of the anatomy and also of the vessels involved. On the other hand, in automatic segmentation of the lesion, the “detection” step may pose significant challenges as the blood vessels and the aneurysm sac may be in the same intensity range. In this work, two different models may be used, (1) a segmentation model or (2) a detection plus segmentation model.

In a segmentation model, a limited area of imaging information, such as a DSA image, containing the aneurysm sac may be received as input. In a semi-automatic segmentation, a pre-processing pipeline may create a rectangle region that is included in the received imaging information. For example, a clinician such as a radiologist may provide minimal input on imaging information such as heuristically padding around the aneurysm sacs. The imaging information, e.g., as marked, annotated, or adjusted by the clinician may be received as input to the segmentation network.

### Detection Plus Segmentation

In a detection plus segmentation model, the image may be received as the input without any interaction/annotation by the clinician, and the neural network model may provide a fully automatic method of detection and segmentation of the aneurysm sac. In the detection plus segmentation model setup the algorithm, may implicitly learn or identify the location of the aneurysm within the image and then perform the segmentation on the identified aneurysm.

### Device Identification/Sizing

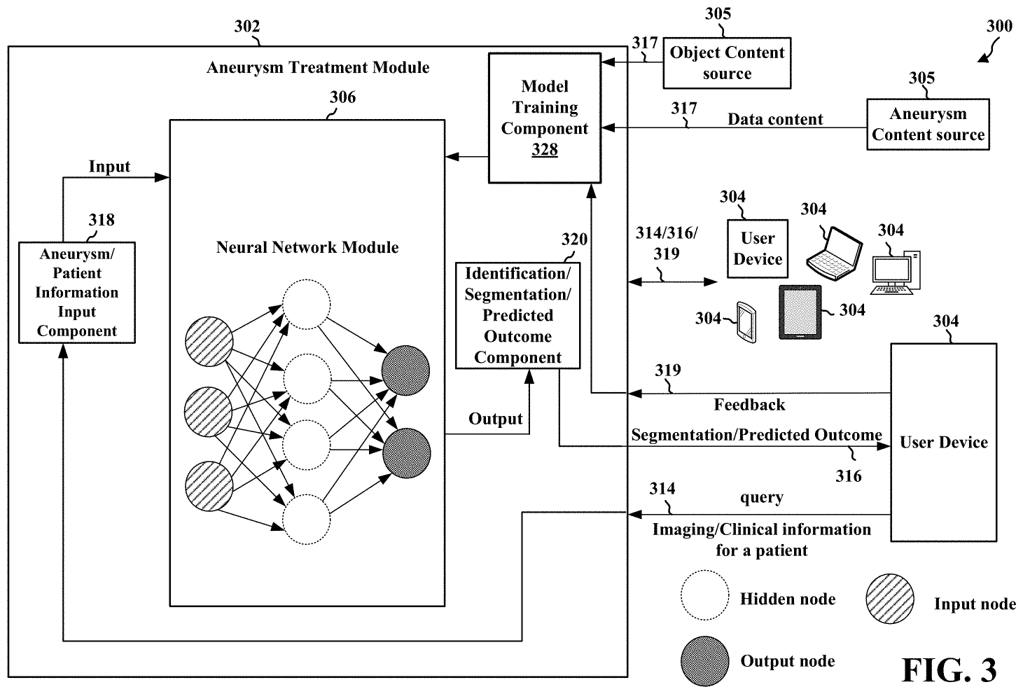

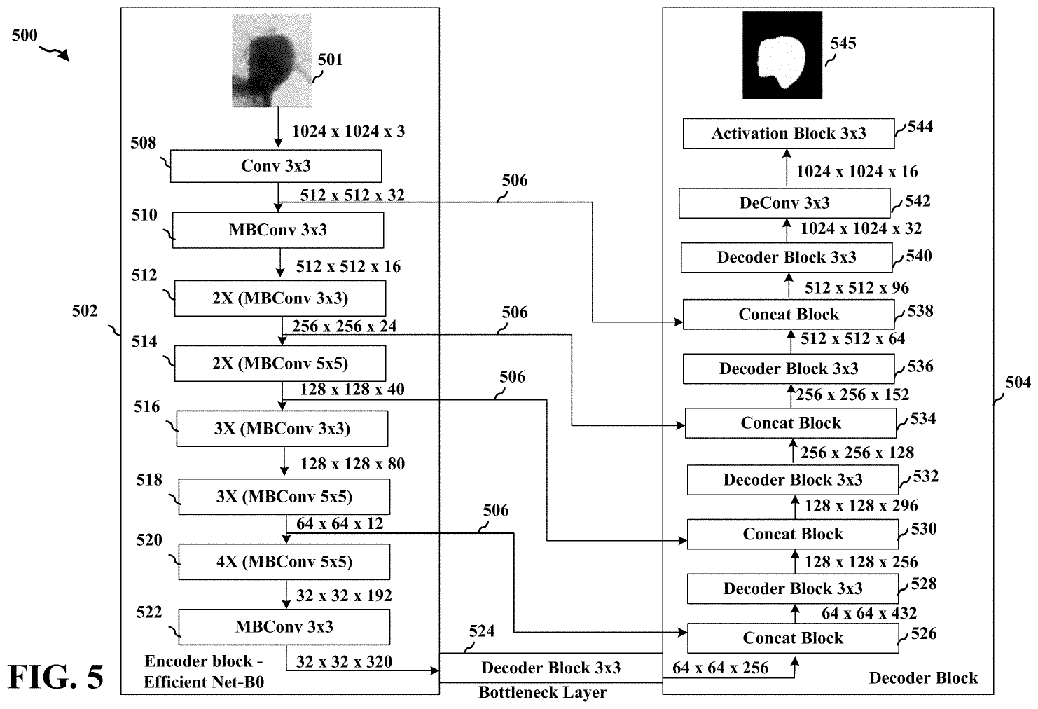

The neural network component 226 may include a treatment device identification component 232 that is configured to output an identification of one or more treatment devices having better predicted outcome than other treatment devices. The treatment device may include one of various intrasaccular embolization devices, for example. As an example, the service may respond to a user device by sending a treatment device size, or a subset or list of treatment devices sizes, that are predicted based on the neural network model to have a higher likelihood of providing a complete occlusion for the aneurysm sac. The neural network component 226 may include an outcome prediction component 234 that outputs a predicted treatment result associated with one or more treatment device sizes. Additional aspects that may be incorporated in the neural network component are described in connection with FIG. 3, FIG. 4C, and FIG. 5.

Aspects presented herein may identify a size of a flow disruptor implant device, such as a size of a WEB implant. The aspects presented herein may provide a predicted outcome for one or more of the flow disruptor implant devices identified as having a higher predicted outcome of complete occlusion than the other analyzed implant devices based on the received information, which may also be referred to as a highest predicted likelihood of a complete occlusion in comparison to a set of potential implant devices. In other aspects, the aspects presented herein may include the neural network or machine learning based analysis and identification of a type, and size, of aneurysm treatment device from multiple types of aneurysm treatment devices based on the imaging study information and/or clinical information for a particular patient. Examples of different aneurysm treatment devices may include a coil embolization type treatment device, a coil assisted stent, other types of stents, a flow disruptor type treatment device, a flow diversion type treatment device, among other example treatment device types. Each of the device types may have models in different sizes, which can each be analyzed according to the neural network model to predict a treatment outcome if the various sizes of the different types of treatment devices were implanted in the aneurysm.

### Outcome Prediction

The machine learning or neural network model may be constructed using clinical and/or imaging features to predict an outcome of treatment. As one example, in order to illustrate the concept, the system may predict an output of treatment for wide-neck bifurcation aneurysms with an intrasaccular embolization device. As illustrated in FIG. 4B, the outcome prediction component 234 may provide, for display to a user, predicted outcomes for various types and/or sizes of devices. The outcome prediction component 234 may suggest to the user a size of the intrasaccular device having the highest likelihood of success, e.g., the highest likelihood of complete occlusion. The outcome prediction component 234 may suggest a size and/or a model, and may also include the likelihood of a full occlusion associated with the suggested size.

The Raymond-Roy Occlusion Classification is a classification system that includes Class I corresponding to a complete occlusion of the aneurysm sac, Class II as a residual neck at of the aneurysm sac, and Class III as a residual aneurysm. Outcomes in aneurysm occlusion can be observed at a time period after a treatment device is implanted in the aneurysm, such as one year after the implant. In some aspects, the predicted outcome may include a predicted percentage of complete occlusion. In some aspects, the predicted outcome may include a predicted classification, e.g., a predicated classification between complete occlusion, partial occlusion, and residual neck. In some aspects, the predicted outcome may include a predicted percentage for one or more classification, e.g., a predicated percentage for complete occlusion, a predicated percentage for partial occlusion, and/or a predicated percentage for residual neck. Various factors may affect the outcome of the aneurysm treatment. As described in connection with FIG. 1C, the size, e.g., including dimensions such as diameter and height, of the treatment device can affect the treatment outcome. As well, relative positions of a parent and daughter vessel in a bifurcation aneurysm can have an impact on aneurysm rupture due to the effect on the blood flow dynamics into the sac region. Other clinical information, such as a history of neurological disorders, gender, and smoking history can be associated with increased incidence of aneurysms. In some aspects, retrospective studies evaluating clinical outcome of aneurysm treatment device implants may be used as training data to train the machine learning model for the automatic prediction of outcomes associated with various treatment devices and sizes of treatment devices.

The module 220 may include a training component 228 that is configured to train and/or update the neural network model applied by the neural network component 226. The training component 228 may be configured to perform any of the aspects described in connection with the model training component 328 in FIG. 3, for example.

Various feature selection approaches may be employed as part of the neural network component 226 and/or the training component 228. As an example, statistical co-occurrence and/or information gain may be used for feature selection. The co-occurrence based approach may be used to identify relevant health conditions from various possible health conditions that may be included as clinical information associated with imaging data and/or outcomes. The selected health conditions may be selected as determinants of outcome based on their differential prevalence in the complete occlusion or partial occlusion class. A ratio of the co-occurrence of a given health condition with the total number of cases for the class. As an example, the ratio of hypertension/complete occlusion ratio may correspond to 28/49=0.57 and hypertension/partial occlusion ratio may correspond to 2819/32=0.59. The ratio of migraines/complete occlusion may correspond to 28/49=0.057 and for migraines/partial occlusion may correspond to 14/32=0.44. Health conditions that have a ratio below a threshold may be filtered out. For example, health conditions having a ratio below 0.30 may be filtered out of the consideration. The threshold may be based on input from a clinician. The health conditions remaining after the filtration correspond to candidate conditions for a classification task. To select discriminative conditions between two classes from candidate conditions, an absolute different may be computed between the prevalence ratios from the two classes. Then, conditions may be retained that have a difference of 0.10 or above. As an example, migraines may be selected that have an absolute difference of 0.13 between the ratios (|0.57−0.11⊕) while hypertension may be filtered out that has an absolute difference of 0.02 between the two ratios (|0.57−0.059|). The information gain may be used as a metric to rank features. The information gain metric may rank features independently for their class separability. The features could have a different rank when used in a set for classification. In order to identify such feature groups, while keeping the top features, e.g., with information gain above 0.15, various combinations of other clinical and imaging features may be added in a greedy fashion based on weighted F1 scores, e.g., in 10-fold cross validation.

FIG. 4C illustrates an example model building workflow 450 that includes receiving imaging data at 452, which may include 2D and/or 3D imaging data. The imaging data may include different types of imaging data, e.g., as shown at 451, 452, and 455. The data may be annotated or unannotated. At 454, measurement extraction is performed. In some aspects, the measurements may be extracted from annotations or may be calculated based on the annotations. In other aspects, the measurements may be based on the imaging data itself without annotations. The measurement information, as well as clinical background and disease history information 456 is input to a classification model 458. Various different types of classification algorithms and combinations of clinical and imaging features may be employed to obtain an output likelihood of complete occlusion, at 460. The outcome may be based various outcome classifications, such as partial versus complete occlusion classification. Various types of classification algorithms may be used in the neural network component 226 or the training component 228. As an example, any combination of random forest algorithms, multilayer perceptron (MLP) neural network, logistic regression, naïve Bayes, or support vector machine (SVM) may be implemented. One or more algorithms may be selected and incorporated into the neural network component 226. As an example, the selection of the algorithm may be based on 10-fold cross-validation and performance measurements, e.g., which may include an F1 score, sensitivity, specificity, and ROC).

Aspects presented herein allow for multiple models and different combinations of clinical and imaging features. Among other examples, Feature set A may include all clinical and pre-operative imaging features and may be used to develop a baseline model. Feature set B may contain no imaging features and instead may include a subset of clinical features that are selected to maximize class separability, e.g., as described above. Feature set C may include the selected clinical features from Feature set B and may add select imaging features, such as from 2D DSA images as calculated directly from clinical annotations. Feature set D may include the same features and Feature set C with the addition of an aneurysm sac volume related measurements extracted from 3D image annotations. Feature set E may replace contour measurements from 2D and volume, surface, and IPR from 3D features with equivalents calculated from automatically delineated contours that were obtained using the neural network. Feature set E may allow for the automatic measurement to be evaluated. The clinical features are the same for Feature Sets B, C, D, and E. Feature set F may allow for a study of the effect of dropping clinical features and may include imaging features from Feature set D without the clinical features. Feature set G may similarly include the imaging features of Feature set E, without the clinical features, and may use automatically computed 3D imaging using a deep neural net segmentation model. The performance of the use of different classification algorithms with different feature sets may be compared and evaluated to determine an algorithm and feature set to be employed in the neural network component 226 and/or the training component 228. The comparison may be based on one or more accuracy, specificity, sensitivity, an F1 score, a weighted F1 score, an F1 score for complete occlusion, an F1 score for partial occlusion, and/or an ROC. The statistical comparison of the prediction results for different feature sets can illustrate the effectiveness on various feature sets and algorithm combinations. Among other examples, a random forest classifier may be used in connection with any combination of feature set. As further described in connection with FIG. 3, the model may be trained, and updated, as additional training data and feedback are provided. The algorithm and/or feature set combination may be changed based on updated data and feedback.

FIG. 3 illustrates a diagram 300 of an aneurysm treatment module 302 that includes a neural network 306, or machine learning component or artificial intelligence component, configured to provide automatic, or semi-automatic, segmentation or treatment outcome predictions for one or more users. In aspects described herein, the machine learning may be accomplished with a neural network, for example. The aneurysm treatment module 302 may have similar or identical functionalities to the aneurysm treatment module 220 in FIG. 2, for example. The aneurysm treatment module 302 may receive information 314 from a user device 304 that provides one or more of automatic or semi-automatic measurements for an aneurysm sac, an identification of a treatment device (e.g., type and/or size) from multiple potential treatment devices, and/or a predicted outcome for one or more aneurysm treatment devices for a particular aneurysm patient based on their imaging study information and/or clinical information. The output may be generated based on a machine learning model, neural network, or artificial intelligence.

FIG. 3 also illustrates that the aneurysm treatment module 302 may receive information 314 from multiple user devices of various types. The information 314 may be based on various different patients. The information input from a user device to the model inference component, which may include a neural network 306. The information may include imaging study data, which may include one or more types of images of an aneurysm of a patient, and which may include measurement information taken from images for the patient. In some aspects, the imaging study information may include images marked or annotated by a clinician. In some aspects, the imaging study information may include non-annotated images, which may be referred to as raw images. The information 314 may include clinical information for the patient, such as any combination of demographic information (e.g., including age, gender, height, weight, race, among other examples of demographic information), aneurysm information (e.g., including location, side, type, manner of detection, grade(s), or score(s), among other examples), allergies of the patient, drugs taken by the patient, pre-existing conditions of the patient (e.g., such as smoking history, substance abuse, affected body systems, among other examples).

Table 2 illustrates various, non-limiting, examples of clinical information that may be provided for a particular patient, at 314. In some aspects, the information 314 may include a query or request for information from the user device 304. The request may be for an automatic or semi-automatic segmentation or measurement of an aneurysm. The request may be for an identification of an optimum subset of treatment devices, treatment device models, or treatment device sizes. The request may be for a prediction of a treatment outcome, which may be associated with one or More treatment devices. The aneurysm treatment module 302 may include any of the components described in connection with FIG. 2, e.g., including a user data interface component 222, a query component 224, a neural network component 226, a training component 228, an identification component 236, a segmentation component 230, a treatment device identification component 232, and/or an outcome prediction component 234.

The neural network 306 may receive the user input information 314 via an input component 318 and may output measurement information for an aneurysm, an identification of one or more aneurysm treatment devices from a group of multiple treatment devices, and/or a predicted outcome of treatment with the one or more identified aneurysm treatment devices. The output 316 may be provided via an output component 320 to a corresponding user device and may presented to the user as a displayed response to the search query at a user interface, transmitted to the user in a message, or provided to the user in some other visual or audio indicator that identifies or flags information about the content at the user device 304.

As illustrated in FIG. 3, the aneurysm treatment module 302 may receive additional information and data 317 from various different sources 305. For example, the model training component 328 may receive training data, e.g., which may include datasets relating to angiographic images with various classifications, e.g., complete occlusion, residual aneurysm, residual neck, etc. The data may be for patients with various clinical information, e.g., including different demographic information. The aneurysm information, clinical information and/or outcome information associated with the image data may be input as training data. Training data may also come from other sources.

The model inference component or neural network 306 may include an image classification neural network. The image classification neural network may include any of a convolutional neural network (CNN), a recurrent neural network (RNN), transfer learning models, and/or a multiplayer perceptrons neural network, among other examples.

As an example, an EfficientNet may be pre-trained on millions of different object images to provide thousands of object classifications. Although the various objects are different than aneurysms, the classification data may be received as input to assist the model in identifying, segmenting, sizing prediction, and/or outcome prediction associated with aneurysms. The model training component 328 may include a data collection component that receives, obtains, and/or prepares data for model training. The preparation of data may include data pre-processing, cleaning, formatting, and transformation. In some aspects, the data received via the model training component may include inference data to be provided as input for the neural network 306. The obtained data may be used as training data to train the neural network 306 in order to provide the output to the user devices 304. The model training component 328 may perform machine learning, or neural network training, validation, and testing. Model performance metrics may be generated as part of the model testing procedure. The model training component 328 may deploy or update a trained, validated, and tested model to the neural network 306 and may receive feedback about the performance of the neural network 306.

In some aspects, one or more user devices 304 may provide feedback 319 to the aneurysm treatment module 302. For example, a user device 304 may provide feedback about a treatment device selected and/or implanted within an aneurysm sac. The user device 304 may provide feedback about a treatment outcome, user rating feedback, etc.

FIG. 3 illustrates that an example neural network 306 may include a network of interconnected nodes. An output of one node is connected as the input to another node. Connections between nodes may be referred to as edges, and weights may be applied to the connections/edges to adjust the output from one node that is applied as the input to another node. Nodes may apply thresholds in order to determine whether, or when, to provide output to a connected node. The output of each node may be calculated as a non-linear function of a sum of the inputs to the node. The neural network 306 may include any number of nodes and any type of connections between nodes. The neural network 306 may include one or more hidden nodes. Nodes may be aggregated into layers, and different layers of the neural network may perform different kinds of transformations on the input. A signal may travel from input at a first layer through the multiple layers of the neural network to output at a last layer of the neural network and may traverse a layer multiple times. As an example, the system may input information from 318 to the neural network 306, and may receive or obtain output (e.g., from 320). The output of may then be provided to a user device 304.

In some aspects, the neural network 306 may use machine-learning algorithms, deep-learning algorithms, neural networks, reinforcement learning, regression, boosting, and/or advanced signal processing methods for receiving content and identifying content of interest for particular users.

In some aspects, unsupervised learning may be used to train the neural network 306. An unsupervised neural network, for example, can learn representations of input that correspond to important characteristics of an input distribution. The neural network may employ algorithms, such as clustering algorithms or distribution algorithms, to analyze and cluster data to discover patterns and data grouping without user intervention, e.g., analyzing training data without added labels or categories, for example. Deep learning algorithms, for example, can identify conclusions and patterns through unlabeled datasets by implicitly learning a distribution function of observed data.

Reinforcement learning is a type of machine learning that involves the concept of taking actions in an environment in order to maximize a reward. Reinforcement learning is a machine learning paradigm. Other paradigms include supervised learning and unsupervised learning. Basic reinforcement may be modeled as a Markov decision process (MDP) with a set of environment states and agent states, as well as a set of actions of the agent. A determination may be made about a likelihood of a state transition based on an action and a reward after the transition. The action selection by an agent may be modeled as a policy. The reinforcement learning may enable the agent to learn an optimal, or nearly-optimal, policy that maximizes a reward. Supervised learning may include learning a function that maps an input to an output based on example input-output pairs, which may be inferred from a set of training data, which may be referred to as training examples. The supervised learning algorithm analyzes the training data and provides an algorithm to map to new examples.

Regression analysis may include statistical analysis to estimate the relationships between a dependent variable (e.g., an outcome variable) and one or more independent variables. Linear regression is an example of a regression analysis. Non-linear regression models may also be used. Regression analysis may include estimating, or determining, relationships of cause between variables in a dataset.

Boosting includes one or more algorithms for reducing variance or bias in supervised learning. Boosting may include iterative learning based on weak classifiers (e.g., that are somewhat correlated with a true classification) with respect to a distribution that is added to a strong classifier (e.g., that is more closely correlated with the true classification) in order to convert weak classifiers to stronger classifiers. The data weights may be readjusted through the process, e.g., related to accuracy.

Among others, examples of machine learning models or neural networks that may be included in the aneurysm treatment module 302 include, for example, artificial neural networks (ANN); decision tree learning; convolutional neural networks (CNNs); deep learning architectures in which an output of a first layer of neurons becomes an input to a second layer of neurons, and so forth; support vector machines (SVM), e.g., including a separating hyperplane (e.g., decision boundary) that categorizes data; regression analysis; Bayesian networks; genetic algorithms; deep convolutional networks (DCNs) configured with additional pooling and normalization layers; and deep belief networks (DBNs).

FIG. 3 illustrates an example machine learning model, such as an artificial neural network (ANN), that includes an interconnected group of artificial neurons (e.g., neuron models) as nodes. Neuron model connections may be modeled as weights, in some aspects. Machine learning models, such as the example in FIG. 3, may provide predictive modeling, adaptive control, and other applications through training via a dataset. A machine learning model may be adapted, e.g., based on external or internal information processed by the machine learning model. In some aspects, a machine learning model may include a non-linear statistical data model and/or a decision making model. Machine learning may model complex relationships between input data and output information.

A machine learning model may include multiple layers and/or operations that may be formed by concatenation of one or more of the referenced operations. Examples of operations that may be involved include extraction of various features of data, convolution operations, fully connected operations that may be activated or deactivated, compression, decompression, quantization, flattening, etc. The term layer may indicate an operation on input data. Weights, biases, coefficients, and operations may be adjusted in order to achieve an output closer to the target output. Weights and biases are examples of parameters of a trained machine learning model. Different layers of a machine learning model may be trained separately.

A variety of connectivity patterns, e.g., including any of feed-forward networks, hierarchical layers, recurrent architectures, feedback connections, etc., may be included in a machine learning model. Layer connections may be fully connected or locally connected. For a fully connected network, a first layer neuron may communicate an output to each neuron in a second layer. Each neuron in the second layer may receive input from each neuron in the first layer. For a locally connected network, a first layer neuron may be connected to a subset of neurons in the second layer, rather than to each neuron of the second layer. A convolutional network may be locally connected and may be configured with shared connection strengths associated with the inputs for each neuron in the second layer. In a locally connected layer of a network, each neuron in a layer may have the same, or a similar, connectivity pattern, yet having different connection strengths.

The machine learning model, artificial intelligence component, or neural network may be trained, such as training based on supervised learning. During training, the machine learning model may be presented with an input that the model uses to compute to produce an output. The actual output may be compared to a target output, and the difference may be used to adjust parameters (e.g., weights, biases, coefficients, etc.) of the machine learning model in order to provide an output closer to the target output. Before training, the output may not be correct or may be less accurate. A difference between the output and the target output, may be used to adjust weights of a machine learning model to align the output is more closely with the target.

A learning algorithm may calculate a gradient vector for adjustment of the weights. The gradient may indicate an amount by which the difference between the output and the target output would increase or decrease if the weight were adjusted. The weights, biases, or coefficients of the model may be adjusted until an achievable error rate stops decreasing or until the error rate has reached a target level.

FIG. 5 is a diagram illustrating an example architecture 500 for a segmentation network, in accordance with the systems and methods described herein. The architecture may be incorporated in the neural network component 226 of FIGS. 2 and 10, as well as the aneurysm treatment module 302 in FIG. 3. The architecture may include an encoder-decoder structure. As an example, the encoder function may change a representation or image (e.g., an image of an aneurysm) into code in a latent space, and the decoder may construct an output based on the code. Fully convolutional deep neural networks such as UNet and MNet may be used for segmenting anatomical structures in medical images. The UNet may include an encoder that acts as a feature generator, followed by a decoder that creates area masks of the size equal to the input image through deconvolution layers. Aspects presented herein further incorporate transfer learning from pre-trained networks, such as an ImageNet, in a model for segmentation.

FIG. 5 illustrates an example in which the encoder 502 is configured to process an image 501 or imaging study information, from one or more types of imaging devices, and the decoder 504 may determine segmentation, e.g., measurement information, representing an aneurysm sac, as shown at 545, based on the code provided from the encoder. A U-net including an encoder function and a decoder function may be structured to perform segmentation on the image. An encoder arm of a U-net structure may include a convolutional neural network trained for classification on a large variety of objects. FIG. 5 illustrates an example in which a trained (e.g., which may be referred to as pre-trained) convolutional neural network is incorporated in the encoder 502. The incorporation of a convolutional neural network may be pre-trained (e.g., on classification or object classification) at the encoder function can improve the segmentation result provided from the decoder 504. The trained convolutional neural network be trained on a large number of images different than the types of images, e.g., to train the model to identify various types of objects of different categories. Among other examples, a family of convolutional neural networks is EfficientNet. As one example, EfficientNet-b0 is a convolutional neural network trained on a large number of different types of objects, e.g., millions images from an image database from categories including common household or inanimate objects, animals, etc. to provide classification of the objects. For example, the weights may be trained on the JFT-300M dataset, for example, that includes 300M images labeled with 18,291 categories. The incorporation of the pre-trained convolutional neural network that is pre-trained for classification can improve the segmentation and measurements of aneurysm sac. In some aspects, the neural network may be run without pre-trained weights.

FIG. 5 illustrates an encoder block (e.g., 502) of a UNet architecture that includes pre-trained EfficientNet-B0 layers and a decoder block (e.g., 504) may include de-convolution blocks trained from scratch. The diagram illustrates skip connects 506 between the encoder block (e.g., 502) and the decoder block (e.g., 504). In the illustrated example of FIG. 5, the encoder block (e.g., 502) includes a 3×3 convolution block 508, a 3×3 MB convolution block 510, a 2×3×3 MB convolution block 512, a 2×5×5 MB convolution block 514, a 3×3×3 MB convolution block 516, a 3×5×5 MB convolution block 518, a 4×5×5 MB convolution block 520, and a 3×3 MB convolution block 522. A 3×3 decoder block 524 may couple the encoder 502 to the decoder 504. The decoder block (e.g., 504) includes a concatenation block 526, a 3×3 decoder block 528, a concatenation block 530, a 3×3 decoder block 532, a concatenation block 534, a 3×3 decoder block 536, a concatenation block 538, a 3×3 decoder block 540, a 3×3 deconvolution block 542, and a 3×3 activation block 544. In the illustrated example, a 3×3 decoder block 524 forms a bottleneck layer between the encoder block (e.g., 502) and the decoder block (e.g., 504).

In an example implementation, the deep learning models may be based on a PyTorch Python package with the network architecture implementation. As an example, to illustrate the concept, each fold may be trained on input image sizes of 1024×1024×3, with batches of 16 images for 20 epochs with an initial learning rate of 1e⁻³ with a reduced learning rate on plateau scheduler with a patience rate of 2 epochs. In some aspects, augmentation including flipping, rotation, and Gaussian blurring may be used to increase the variety of training images.

As shown by Table 3, the incorporation of the convolutional neural network at the encoder, e.g., and the further use of a pre-trained convolutional neural network, provides an improved Dice coefficient (2× intersection/union) in comparison to a clinician marked aneurysm. When two contours completely overlap, the Dice coefficient equals the maximum of 1.

TABLE 3

| Method | Task | Dice |
|---|---|---|
| Baseline: UNet | Segmentation (e.g., of a | 0.628 ± 0.216 |
| | marked or limited area of an | |
| | image) | |
| UNet, EfficientNet-B0 | Segmentation (e.g., of a | 0.864 ± 0.091 |
| encoder (w/o pre-train) | marked or limited area of an | |
| | image) | |
| UNet, EfficientNet-B0 | Segmentation (e.g., of a | 0.904 ± 0.062 |
| encoder (with pre-train) | marked or limited area of an | |
| | image) | |
| UNet, EfficientNet-B0 | detection plus segmentation | 0.767 ± 0.267 |
| encoder (with pre-train) | | |
| Baseline 3D | 3D Segmentation | 0.675 (0.620- |
| | | 0.730 |
| UNet, EfficientNet-B1 | 3D Segmentation, slice-by- | 0.810 (0.775- |
| encoder | slice | 0.844) |
| AHNet 3D, ResNet50 | 3D segmentation | 0.836 (0.813- |
| encoder | | 0.859) |

The tool may be used to detect, segment, or measure aneurysms in various locations, such as aneurysms in basilar artery, middle cerebral artery, anterior communication artery, or internal carotid artery (carotid terminus).

As Table 3 shows, when the proposed network works in the semi-automatic settings on the segmentation only problem, the average of the Dice coefficient over the fifteen folds is 0.904 with a standard deviation of 0.062. The EfficientNet encoder based UNet segmentation may be run without the pre-trained training, from scratch and achieved a score of 0.864±0.091 (Row 2, Table 3). In the results in Table 3, the architecture and pre-trained weights of EfficientNet provide improved results. In addition to showing a higher accuracy compared to a UNet architecture, which has not been shown for a VGG architecture pre-trained on ImageNet as a pixel-by-pixel classifier or a UNet, the aspects presented are different than detection and segmentation with a UNet structure along with an LTSM architecture to model the changes in the image as contrast agent is introduced. Table 3 also illustrates results for 3D segmentation using a neural network, as described herein.

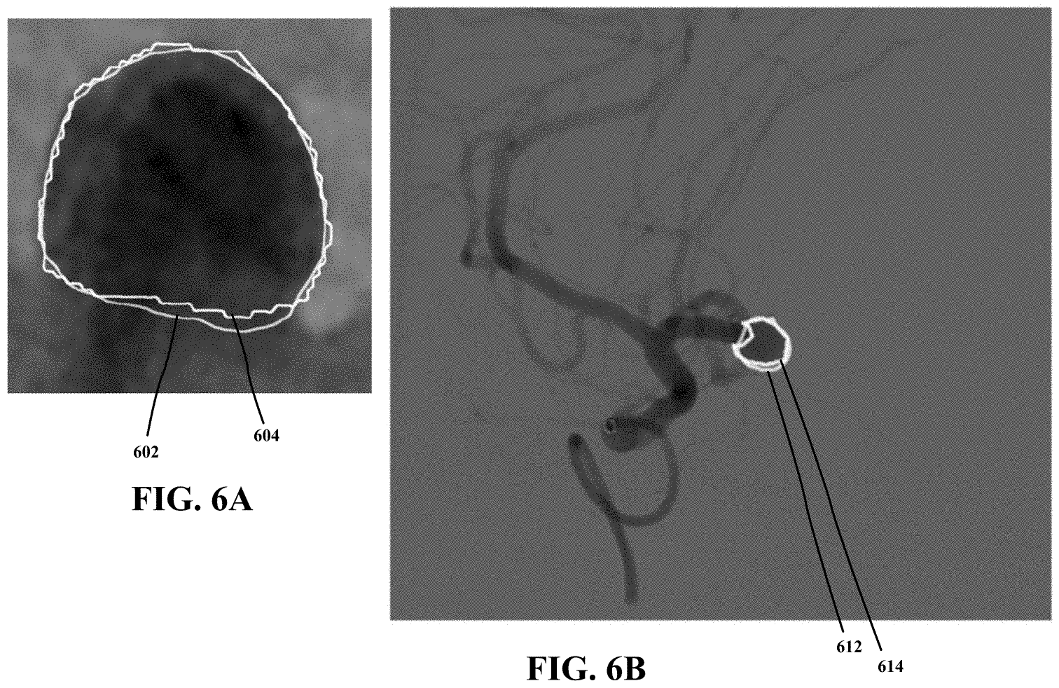

FIG. 6A is an illustration showing an outline 602 based on segmentation of detected aneurysm sac using a model structure described in connection with FIG. 5. A second line 604, shows segmentation with a UNet without the EfficientNet encoder arm, which may be referred to as a ground truth contour for the comparison. FIG. 6A illustrates the improved segmentation on the aneurysm image provided by the model structure in FIG. 5. FIG. 6B is a diagram illustrating detection and segmentation of an aneurysm sac with the proposed network. A first line 612 illustrates the aneurysm detected and segmented with the architecture in FIG. 5, and the second line 614 illustrates a ground truth contour for comparison. The segmentation approach proposed here provides a reliable automatic measurement similar to the clinical practice that uses 2D images. The robustness of the method is evident from the fact that the neural network produced accurate contours even in raw angiograms, as shown in FIG. 6B.

The detection plus segmentation network provides an alternative semi-automatic approach to marking the aneurysm sacs that allows the full image to be provided as input to the network. The automatic identification and segmentation may be provided to a user, such as an INR, who can accept or reject the contour provided by the neural network. In some aspects, if the user rejects the detection and segmentation result, the user can provide some additional input, such as to mark the region of the sac, and the image can be segmented by the segmentation only network or function rather than the identification and segmentation function. In some aspects, if a detection and/or segmentation is rejected by the user, the rejection may be provided as feedback to a model training component, such as shown at 319 in FIG. 3.

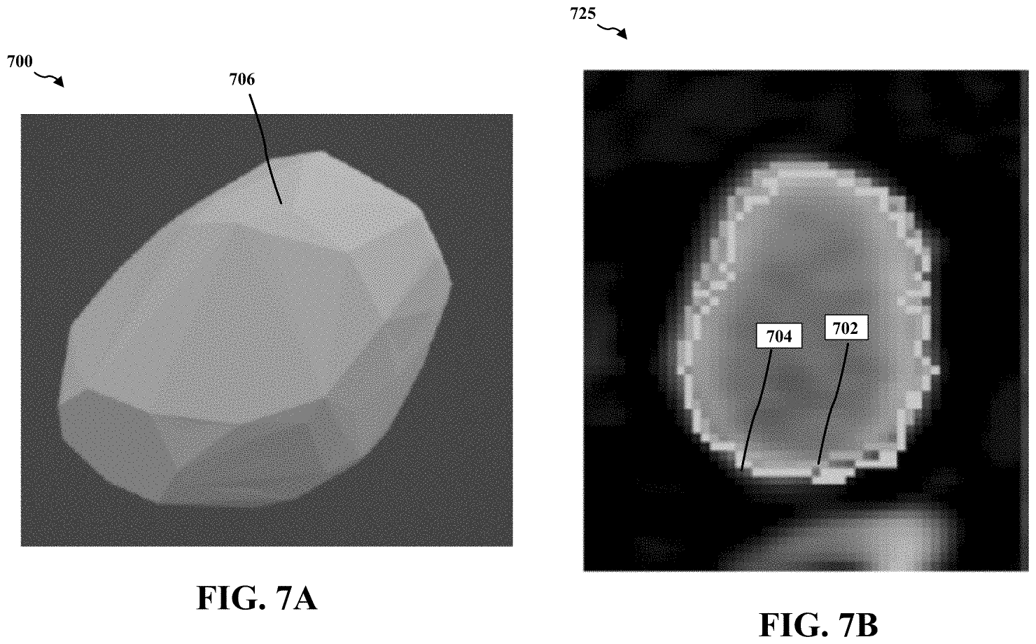

In addition to a 2D segmentation algorithm, a 3D axial stack segmentation algorithm may be employed. In such aspects, an extended version of a 3D UNet may be used, e.g., an AH-Net, to segment the 3D data. In an example, the architecture may include a pre-trained ResNet 50 architecture, as one example, to generate the encoder features. The feature decoder may use anisotropic 3D convolutions to learn contextual information across the 2D slice-by-slice features generated from the encoder. This allows the network to leverage well trained 2D feature generators even for complex 3D tasks. The pre-processing pipeline for a semi-automated extraction of a heuristically determined cube around the aneurysm may be based on some input from a user, such as a radiologist. As one example to illustrate the concept, the pipeline may include a PyTorch with an AHNet architecture implementation. Weak augmentations of randomaffine and/or randomanisotropy may be used. A k-fold cross validation (k=15, train: 38.27±0.44, valid: 2.73±0.44) may be performed on image sizes 60×60×60, with batches of 12 for 30 epochs with a learning rate of 1e⁻³. A Dice coefficient loss may be used in connection with the deep learning method to optimize the network. Table 3 includes some example results for 3D segmentation perform based on the aspects presented herein. FIG. 7A illustrates an example image 700 of a 3D mesh model 706 of an aneurysm sac reconstructed from segmented contours, which may be input to the model of the present disclosure. FIG. 7B illustrates an example result 725 of the proposed 3D neural network for segmentation of a detected aneurysm. The line 702 shows the contour output by the automated system, and the line 704 illustrates a ground truth or baseline contour, e.g., corresponding to the 3D baseline in Table 3.

The accurate measurement of the dimensions of the aneurysm sac is an important step in treatment planning for cerebral aneurysms. Aspects presented herein provide a semi-automatic, and a fully automatic method for segmenting the sac based on imaging study information and/or clinical information input to a machine learning model. Aspects additionally provide for the automatic identification of a treatment device or a set of potential treatment devices to be implanted in the aneurysm sac. Aspects additionally provide for a prediction of an outcome result associated with the identified treatment device or set of treatment devices. The decision support software systems provide a tool to assist in selecting a promising treatment at a rapid pace. The automated detection and/or segmentation, the identification of a potential treatment device (e.g., including a size or model), and/or the information about a predicted outcome of such treatment provides added tools for a user to consider in developing a treatment plan. The automated aspects reduce the time for identification, segmentation, and identification of treatment devices, which can increase the pace at which treatment can be delivered to a patient. As well, the use of a neural network or machine learning component enables the added consideration of a history of other data including prior aneurysm data in performing the detection, identification, and segmentation. The feedback aspects enable the machine learning model to continually improve the output provided to users. The combined use of imaging data and clinical information provides a more accurate and efficient estimation or prediction. The added efficiency and accuracy provide a tool that assists users in developing treatment plans.

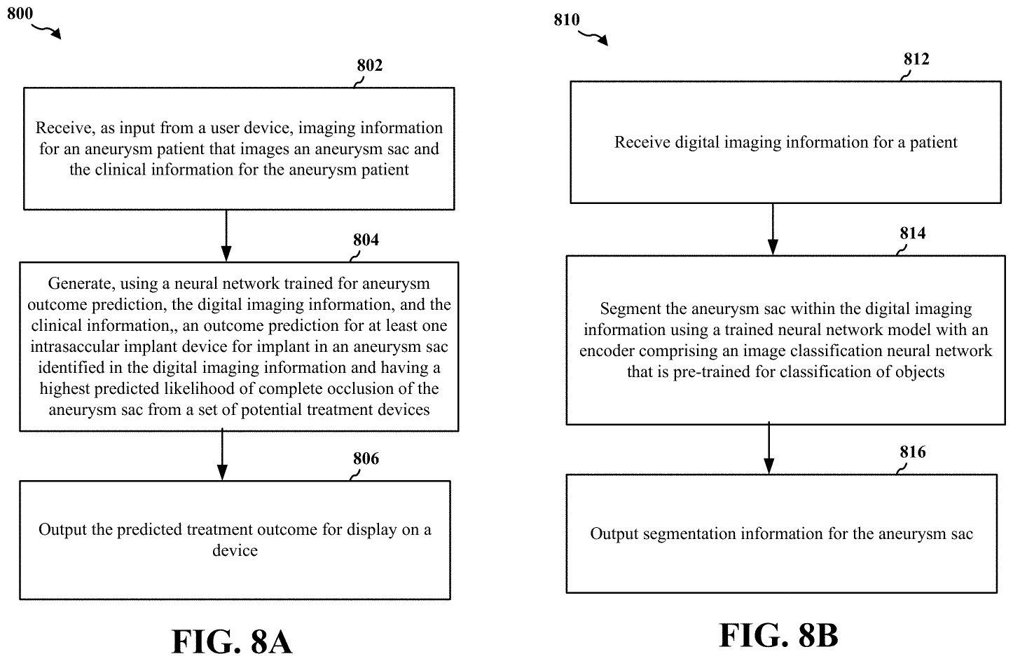

FIG. 8A illustrates a flowchart 800 for a method or algorithm for providing, from a neural network apparatus, outcome predictions for intrasaccular implant devices based on images (e.g., angiography imaging) and clinical information. The neural network apparatus may correspond to the aneurysm treatment module 220 in FIG. 2 or 302 in FIG. 3, or may comprise the aneurysm assistance component 1075 in FIG. 10.

At 802, the neural network apparatus receives, as input from a user device, digital imaging information and the clinical information for the aneurysm patient. As an example, the input may be received via one or more of a user data interface component 222 or a query component 224 of an aneurysm treatment module 220. The input may be received at a user interface, such as described in connection with FIG. 4A, for example. In some aspects, the input may be received directly from an image measurement or acquisition device, as shown in FIG. 2. In some aspects, the neural network may be comprised in an image acquisition or measurement device. In some aspects, images, anatomical, and/or clinical data may be uploaded to module 220/302 for use as the encoder inputs. In some aspects, the input may be received as a user query for outcome prediction information.

At 804, the neural network apparatus generates an outcome prediction for at least one intrasaccular implant device for implant in an aneurysm sac identified in the digital imaging information, e.g., the implant device identified as having a highest predicted likelihood of complete occlusion of the aneurysm sac from a set of potential treatment devices. The outcome prediction is generated using a neural network trained for aneurysm outcome prediction, the digital imaging information, and the clinical information. The neural network may include an encoder comprising an image classification neural network (e.g., an image classification neural network may include a convolutional neural network or other types of NN such as RNN, transfer learning models, and/or multiplayer perceptrons neural network) that is pre-trained for classification of objects such as, but not limited to, vessel walls and aneurysms. The outcome prediction is generated by the neural network, which can include a size or model of an intrasaccular device having the highest predicted likelihood for complete occlusion of the aneurysm sac based on the received digital imaging information and/or the clinical information. An intrasaccular device having a highest predicted likelihood for complete occlusion can be a device with the highest percentage of achieving complete occlusion as compared to the percentages of other devices considered by the model. Alternatively, the outcome prediction generated by the neural network can include a list of sizes of an intrasaccular device and the corresponding likelihood of complete occlusion associated with each size.

The neural network may be configured with a classification algorithm based on at least one of a random forest algorithm, an MLP neural network algorithm, a logistic regression algorithm, a naive Bayes machine learning algorithm, or a SVM algorithm. The outcome prediction may be generated, e.g., by the neural network component 226, the outcome prediction component 234 or the neural network 306, as an example. The generation may further include any of the aspects described in connection with FIGS. 1A-7B.

At 806, the neural network apparatus outputs, for display at a device in response to the input from the user device, an identification of the at least one intrasaccular implant device and the outcome prediction for each of the at least one intrasaccular implant device. The device may be a display at the user device that sent input or may be an additional or separate device. As an example, the output may be provided to the user device via a user data interface component 222. The output may be presented at a display to a user of the user device. An example of a user interface displaying output at a user device is illustrated in FIG. 4B.

In some aspects, the neural network apparatus may be further configured to perform semi-automatic segmentation of the digital imaging information based on an annotation in the digital imaging information to obtain one or more measurements of the aneurysm sac by passing the digital imaging information through the encoder to obtain code and through the decoder to output the one or more measurements of the aneurysm sac based on the code.

In some aspects, the neural network apparatus may be further configured to perform automatic segmentation of raw imaging information to identify the aneurysm sac and to obtain the one or more measurements of the aneurysm sac by passing the digital imaging information through the encoder to obtain the code and through the decoder to output the one or more measurements of the aneurysm sac based on the code.

The outcome prediction for each of the at least one intrasaccular implant device may be based on the one or more measurements obtained for the aneurysm sac, dimensions of the at least one intrasaccular implant device, and the clinical information for the aneurysm patient.

FIG. 8B illustrates a flowchart 810 for a method or algorithm for providing, from a neural network apparatus, segmentation information obtained for an aneurysm sac. The neural network apparatus may correspond to the aneurysm treatment module 220 in FIG. 2 or 302 in FIG. 3, or may comprise the aneurysm assistance component 1075 in FIG. 10.

At 812, the neural network apparatus receives digital imaging information (e.g., CT images, ultrasound images, among other examples) for a patient. In some aspects, the digital information can be angiography data. The image classification neural network (e.g., CNN, RNN, among other examples) may be pre-trained for the classification of objects different than aneurysms. The input may be received at a user interface, such as described in connection with FIG. 4A, for example. As an example, the input may be received via one or more of a user data interface component 222 or a query component 224 of an aneurysm treatment module 220.

Examples of a neural network architecture are described in connection with FIG. 5. The trained neural network model may further comprise a decoder trained from scratch to segment aneurysm sacs in images and configured to output measurement information for the aneurysm sac after the digital information is processed at the encoder. The digital imaging information may include one or more 2D DSA images of a wide-neck bifurcation aneurysm prior to implantation of an intrasaccular device. The digital information may include one or more of a lateral and AP views on a 2D DSA image or a 3D axial slice stack reconstructed from a sequence of DSA images.

At 814, the neural network apparatus segments the aneurysm sac within the digital imaging information using a trained neural network model with an encoder comprising an object classification neural network (e.g., CNN, RNN, among other examples) that is pre-trained for classification of objects. The digital information may include an annotation or adjustment identifying the aneurysm sac, and the neural network apparatus may semi-automatically segment the aneurysm sac using the trained neural network model. The digital information may include a raw angiography image, and the neural network apparatus may identify a presence of the aneurysm sac in the raw angiography image using the trained neural network model prior to automatic segmentation of the aneurysm sac. The segmentation may be performed by the neural network component 226, the outcome prediction component 234 or the neural network 306, as an example. The neural network apparatus may further perform any of the aspects described in connection with FIGS. 1A-7B.

At 816, the neural network apparatus outputs segmentation information for the aneurysm sac. As an example, the output may be provided to the user device via a user data interface component 222. The output may be presented at a display to a user of the user device or to a display of a different, additional device.

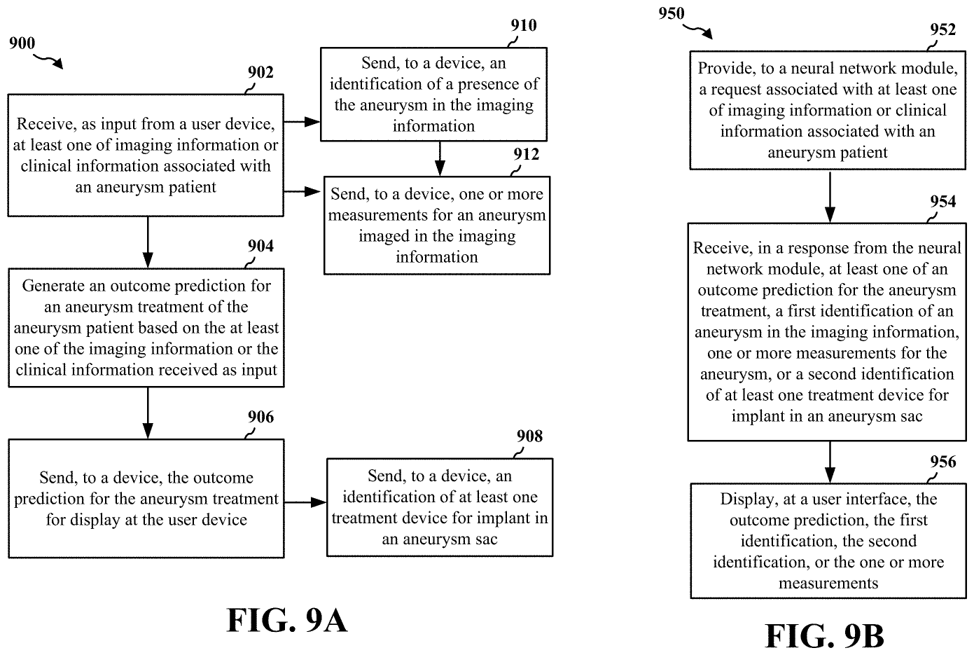

FIG. 9A illustrates a flowchart 900 for a method or algorithm for providing outcome predictions for intrasaccular implant devices based on digital imaging information and clinical information. The method may be performed by a system or service, which may correspond to the aneurysm treatment module 220 in FIG. 2 or 302 in FIG. 3, or may comprise the aneurysm assistance component 1075 in FIG. 10.

At 902, the system receives, as input from a user device, at least one of imaging information or the clinical information associated with an aneurysm patient. As an example, the input may be received via one or more of a user data interface component 222 or a query component 224 of an aneurysm treatment module 220. The input may be received at a user interface, such as described in connection with FIG. 4A, for example. In some aspects, the input may be received directly from an image measurement or acquisition device, as shown in FIG. 2. In some aspects, the neural network may be comprised in an image acquisition or measurement device. In some aspects, the input may be received as a user query for outcome prediction information.

The input may include both the imaging information and the clinical information associated with the aneurysm patient and the system may generate the outcome prediction for the aneurysm treatment based on both the imaging information and the clinical information. The clinical information may include at least one of demographic information for the aneurysm patient, aneurysm information associated with the imaging information, dimension information for an aneurysm imaged in the imaging information, allergies of the aneurysm patient, medication information for the aneurysm patient, or pre-existing condition information for the aneurysm patient. Various examples of clinical information are described in connection with Table 2, for example. The imaging information may include one or more of MRI information, MRA information, a CT scan information, 2D DSA information, or a 3D reconstruction from a sequence of 2D images.

In some aspects, the input may include only the imaging information and the system may generate the outcome prediction for the aneurysm treatment based only on imaging information.

At 904, the system generates an outcome prediction for an aneurysm treatment of the aneurysm patient based on the at least one of the imaging information or the clinical information received as input. The system may include a neural network trained for aneurysm outcome prediction, such as described in connection with any of FIG. 2, 3, 4C, or 5. The system may process the imaging information and the clinical information using the neural network to generate the outcome prediction for the aneurysm treatment. The neural network may include an encoder and decoder, the encoder comprising a convolutional neural network that is pre-trained for classification of objects. The neural network may be configured with a classification algorithm based on at least one of a random forest algorithm, an MLP neural network algorithm, a logistic regression algorithm, a naive Bayes machine learning algorithm, or an SVM algorithm.

At 906, the system sends, to the user device, the outcome prediction for the aneurysm treatment for display at on a device. The device may be a display at the user device that sent input or may be an additional or separate device. The outcome prediction may be associated with a treatment device, which may include an intrasaccular implant device. There may be a set of potential intrasaccular implant devices of different sizes and/or types. The system may identify a subset of one or more devices having a higher likelihood of a complete occlusion outcome after being implanted in the aneurysm sac. The outcome prediction may indicate a predicted likelihood of complete occlusion of an aneurysm sac imaged in the imaging information. The system may output the best predicted sizing having the highest percentage of success, e.g., of complete occlusion of the aneurysm sac. As an example, the output may be provided to the user device via a user data interface component 222. The output may be presented at a display to a user of the user device. An example of a user interface displaying output at a user device is illustrated in FIG. 4B.

As illustrated at 912, the system may be further configured to send, to a device (e.g., for display at the device), one or more measurements for an aneurysm imaged in the imaging information. The device may be a display at the user device that sent input or may be an additional or separate device. For example, the system may be configured to perform segmentation, such as described in connection with any of FIG. 2, 3, 5, or 8B. The segmentation may comprise semi-automatic segmentation and/or automatic segmentation and may include defining a contour surrounding the image of the aneurysm sac. For example, the imaging information may include at least one annotation identifying a region of the aneurysm, and the segmentation may include semi-automatic segmentation in response to reception of the input image information. As another example, the imaging information includes raw imaging information, and the system may be configured to send, to a device, an identification of a presence of the aneurysm in the imaging information in addition to the one or more measurements, e.g., as illustrated at 910. The identification may include a contour outlining an aneurysm sac imaged in the imaging information. Following the identification, the system may send, to a device, the one or more measurements, e.g., which may be referred to as automatic segmentation, at 912. The device may be a display at the user device that sent input or may be an additional or separate device. The outcome prediction, at 906, may include one or more measurements and/or a best predicted size of an intrasaccular device for the aneurysm imaged in the imaging information.

As illustrated at 908, the system may send, to a user device, an identification of at least one treatment device for implant in an aneurysm sac of the aneurysm patient based on the at least one of the imaging information or the clinical information for the aneurysm patient, the at least one treatment device identified based on having a higher predicted likelihood of complete occlusion of the aneurysm sac imaged in the imaging information for the aneurysm patient than other treatment devices in a set of potential treatment devices. The device may be a display at the user device that sent input or may be an additional or separate device. In some aspects, the identification may include a list of multiple treatment devices and a respective outcome prediction associated with each treatment device in the list of multiple treatment devices. FIG. 4B illustrates an example user interface displaying identified treatment devices and an associated outcome prediction. In some aspects, the multiple treatment devices may include different sizes of a same type of intrasaccular implant device having a most favorable outcome prediction from the set of potential treatment devices. The multiple treatment devices may include different types of aneurysm treatment devices.

The algorithm may further include any of the aspects described in connection with FIG. 4C, 8A or 8B.

FIG. 9B illustrates a flowchart 950 for a method or algorithm for obtaining outcome predictions for intrasaccular implant devices based on imaging and clinical information for an aneurysm patient. The method may be performed by a user device, which may correspond to a user device 204 in FIG. 2 or 304 in FIG. 3, for example. At 952, the user device provides, to a neural network module (e.g., such as the module 220 or 302) a request associated with at least one of digital imaging information or clinical information for an aneurysm patient. In some aspects, the request may be based on sending the digital imaging device or clinical information to the neural network module. In other aspects, the request may be sent in response to a user selection at a user interface of the user device, e.g., as described in connection with FIG. 4A.

At 954, the user device receives, in a response from the neural network module, at least one of an outcome prediction for the aneurysm treatment, a first identification of an aneurysm in the imaging information, one or more measurements for the aneurysm, or a second identification of at least one treatment device for implant in an aneurysm sac. The response may include any of the information described in connection with the information sent to the device at 316, 806, 816, 906, 908, 910, and/or 912, for example.

At 956, the user device may further display, at a user interface, the outcome prediction, the first identification, the second identification, or the one or more measurements. FIG. 4B illustrates an example of aspects that may be presented at a user interface. In some aspects, a user may accept or reject one or more aspects provided as outcome. In some aspects, the user interface may include a feature to allow the user to input the acceptance or rejection of the information, such as an identification or measurement of an aneurysm sac. The user input may be provided to the neural network model as feedback.

FIG. 10 is a block diagram illustrating an example computer system 1020 on which aspects of systems and methods for providing segmentation on an aneurysm image, identification of aneurysms, and outcome prediction using a neural network, e.g., as disclosed herein. The computer system 1020 can correspond to the physical server(s) on which an application 1017 is executing, in some aspects. The computer system 1020 may be a part of or may comprise an imaging apparatus that acquires imaging study information for aneurysms. The computer system may be coupled to, e.g., either directly or via a local network or internet network to a device for acquiring such imaging data. In some aspects, the computer system may be coupled to, e.g., either directly or via a local network or internet network, to a user device. The computer system 1020 may include an aneurysm assistance component 1075 configured to perform the aspects described in connection with FIGS. 1A-9A. As an example, the neural network component 226 may be configured to perform any of the aspects described in connection with the algorithm in in FIG. 4C, FIG. 5, FIG. 8A, FIG. 8B, and/or FIG. 9A, as well as any of the aspects described in connection with the aneurysm treatment module 220 in FIG. 2 or 302 in FIG. 3, and/or to receive the input described in FIG. 4A, or provide any of the output described in connection with FIG. 4B, FIG. 6A, 6B, 7A, or 7B. In some aspects, one or more aspects of the computer system 1000 may be configured to perform the aspects described in connection with FIG. 9B or performed by the user device 204 and/or 304

As shown, the computer system 1020 (which may be a personal computer or a server) includes a central processing unit 1021, a system memory 1022, and a system bus 1023 connecting the various system components, including the memory associated with the central processing unit 1021. As will be appreciated by those of ordinary skill in the art, the system bus 1023 may comprise a bus memory or bus memory controller, a peripheral bus, and a local bus that is able to interact with any other bus architecture. The system memory may include permanent memory (ROM) 1024 and random-access memory (RAM) 1025. The basic input/output system (BIOS) 1026 may store the basic procedures for transfer of information between elements of the computer system 1020, such as those at the time of loading the operating system with the use of the ROM 1024.

The computer system 1020 may also comprise a hard disk 1027 for reading and writing data, a magnetic disk drive 1028 for reading and writing on removable magnetic disks 1029, and an optical drive 1030 for reading and writing removable optical disks 1031, such as CD-ROM, DVD-ROM and other optical media. The hard disk 1027, the magnetic disk drive 1028, and the optical drive 1030 are connected to the system bus 1023 across the hard disk interface 1032, the magnetic disk interface 1033, and the optical drive interface 1034, respectively. The drives and the corresponding computer information media are power-independent modules for storage of computer instructions, data structures, program modules, and other data of the computer system 1020.

An example aspect comprises a system that uses a hard disk 1027, a removable magnetic disk 1029 and a removable optical disk 1031 connected to the system bus 1023 via the controller 1055. It will be understood by those of ordinary skill in the art that any type of media 1056 that is able to store data in a form readable by a computer (solid state drives, flash memory cards, digital disks, random-access memory (RAM) and so on) may also be utilized.

The computer system 1020 has a file system 1036, in which the operating system 1035 may be stored, as well as additional program applications 1037, other program modules 1038, and program data 1039. A user of the computer system 1020 may enter commands and information using keyboard 1040, mouse 1042, or any other input device known to those of ordinary skill in the art, such as, but not limited to, a microphone, joystick, game controller, scanner, etc. Such input devices typically plug into the computer system 1020 through a serial port 1046, which in turn is connected to the system bus, but those of ordinary skill in the art will appreciate that input devices may be also be connected in other ways, such as, without limitation, via a parallel port, a game port, or a universal serial bus (USB). A monitor 1047 or other type of display device may also be connected to the system bus 1023 across an interface, such as a video adapter 1048. In addition to the monitor 1047, the personal computer may be equipped with other peripheral output devices (not shown), such as loudspeakers, a printer, etc.

Computer system 1020 may operate in a network environment, using a network connection to one or more remote computers 1049. The remote computer (or computers) 1049 may be local computer workstations or servers comprising most or all of the aforementioned elements in describing the nature of a computer system 1020, e.g., and may include applications 1037 and 1037′. Other devices may also be present in the computer network, such as, but not limited to, routers, network stations, peer devices or other network nodes.

Network connections can form a local-area computer network (LAN) 1050 and a wide-area computer network (WAN). Such networks are used in corporate computer networks and internal company networks, and they generally have access to the Internet. In LAN or WAN networks, the computer system 1020 is connected to the local-area network 1050 across a network adapter or network interface 1051. When networks are used, the computer system 1020 may employ a modem 1054 or other modules well known to those of ordinary skill in the art that enable communications with a wide-area computer network such as the Internet. The modem 1054, which may be an internal or external device, may be connected to the system bus 1023 by a serial port 1046. It will be appreciated by those of ordinary skill in the art that said network connections are non-limiting examples of numerous well-understood ways of establishing a connection by one computer to another using communication modules.

In various aspects, the systems and methods described herein may be implemented in hardware, software, firmware, or any combination thereof. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general purpose computer.

In various aspects, the systems and methods described in the present disclosure can be addressed in terms of modules. The term “module” as used herein refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement the module's functionality, which (while being executed) transform the microprocessor system into a special-purpose device. A module may also be implemented as a combination of the two, with particular functions facilitated by hardware alone, and other functions facilitated by a combination of hardware and software. In particular implementations, at least a portion, and in some cases, all, of a module may be executed on the processor of a general purpose computer. Accordingly, each module may be realized in a variety of suitable configurations, and should not be limited to any particular implementation exemplified herein.

In one configuration, the aneurysm assistance component 1075 and/or the computer system 1020, and in particular, the file system 1036 and/or the processor (e.g., 1021), or one or more of the interfaces provide means for performing any of the aspects described in connection with the flowcharts in FIG. 4C, FIG. 5, FIG. 8A, FIG. 8B, and/or FIG. 9A, as well as any of the aspects described in connection with the aneurysm treatment module 220 in FIG. 2 or 302 in FIG. 3, and/or to receive the input described in FIG. 4A, or provide any of the output described in connection with FIG. 4B, FIG. 6A, 6B, 7A, or 7B. In some aspects, one or more aspects of the system 1000 may be configured to perform the aspects described in connection with FIG. 9B or performed by the user device 204 and/or 304, and may correspond to means for performing any of the aspects described in connection with FIG. 9B and user device 204 and/or 304.

One or more elements or aspects or steps, or any portion(s) thereof, from one or more of any of the systems and methods described herein may be combined with one or more elements or aspects or steps, or any portion(s) thereof, from one or more of any of the other systems and methods described herein and combinations thereof, to form one or more additional implementations and/or claims of the present disclosure.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

Some portions of the detailed description are presented in terms of algorithms and symbolic representations of operations on data bits within a computer memory. These algorithmic descriptions and representations are the methods used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared or otherwise manipulated. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following disclosure, it is appreciated that throughout the disclosure terms such as “processing,” “computing,” “calculating,” “determining,” “displaying” or the like, refer to the action and processes of a computer system, or similar electronic computing device, that manipulates and transforms data represented as physical (electronic) quantities within the computer system's registers and memories into other data similarly represented as physical quantities within the computer system's memories or registers or other such information storage, transmission or display.

Finally, the algorithms and displays presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. It will be appreciated that a variety of programming languages may be used to implement the teachings of the invention as described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

Furthermore, as will be apparent to one of ordinary skill in the relevant art, the modules, routines, features, attributes, methodologies and other aspects of the present invention can be implemented as software, hardware, firmware or any combination of the three. Also, wherever a component, an example of which is a module, of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of ordinary skill in the art of computer programming.

Additionally, the present invention is in no way limited to implementation in any specific programming language, or for any specific operating system or environment. Accordingly, the disclosure of the present invention is intended to be illustrative, but not limiting, of the scope of the present invention, which is set forth in the following claims.

It is understood that the specific order or hierarchy of blocks in the processes/flowcharts disclosed is an illustration of example approaches. Based upon design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

The following aspects are illustrative only and may be combined with other aspects or teachings described herein, without limitation.

Aspect 1 is a method for providing outcome predictions for intrasaccular implant devices at a neural network apparatus based on digital imaging and clinical information, the method including receiving, as input from a user device, digital imaging information and the clinical information for an aneurysm patient; generating, using a neural network trained for aneurysm outcome prediction, the digital imaging information, and the clinical information, an outcome prediction for at least one intrasaccular implant device for implant in an aneurysm sac identified in the digital imaging information and having a highest predicted likelihood of complete occlusion of the aneurysm sac from a set of potential treatment devices; and outputting, for display on a device, an identification of the at least one intrasaccular implant device and the outcome prediction for each of the at least one intrasaccular implant device.

In aspect 2, the method of aspect 1 further includes that the neural network is configured with a classification algorithm based on at least one of a random forest algorithm, a multilayer perceptron (MLP) neural network algorithm, a logistic regression algorithm, a naive Bayes machine learning algorithm, or a support vector machine (SVM) algorithm.

In aspect 3, the method of aspect 1 or aspect 2 further includes performing at least one of: semi-automatic segmentation of the digital imaging information to obtain one or more measurements of the aneurysm sac by passing the digital imaging information through an encoder to obtain code and through a decoder to output the one or more measurements of the aneurysm sac based on the code, or automatic segmentation of raw imaging information to identify the aneurysm sac and to obtain the one or more measurements of the aneurysm sac by passing the digital imaging information through the encoder to obtain the code and through the decoder to output the one or more measurements of the aneurysm sac based on the code, wherein the outcome prediction for each of the at least one intrasaccular implant device is based on the one or more measurements obtained for the aneurysm sac, dimensions of the at least one intrasaccular implant device, and the clinical information for the aneurysm patient.

Aspect 4 is a neural network apparatus for providing outcome predictions for intrasaccular implant devices based on digital imaging and clinical information, the apparatus including memory; and at least one processor coupled to the memory, and based at least in part on information stored in the memory, configured to the method of any of aspects 1-3.

Aspect 5 is a neural network apparatus for providing outcome predictions for intrasaccular implant devices based on digital imaging and clinical information, the apparatus including means for performing the method of any of aspects 1-3.

Aspect 6 is a non-transitory computer-readable storage medium storing computer executable code for providing outcome predictions for intrasaccular implant devices at a neural network apparatus based on digital imaging and clinical information, the code when executed by a processor causes the processor to perform the method of any of aspects 1-3.

Aspect 7 is a method for providing outcome predictions for intrasaccular implant devices at a system based on imaging and clinical information, including receiving, as input from a user device, at least one of imaging information or the clinical information associated with an aneurysm patient; generating an outcome prediction for an aneurysm treatment of the aneurysm patient based on the at least one of the imaging information or the clinical information received as the input; and sending, to a display, the outcome prediction for the aneurysm treatment for display at the user device.

In aspect 8, the system includes a neural network trained for aneurysm outcome prediction, and the method of aspect 7 further includes processing the imaging information and the clinical information using the neural network to generate the outcome prediction for the aneurysm treatment.

In aspect 9, the method of aspect 7 or aspect 8 further includes that the neural network includes an encoder and decoder, the encoder comprising an image classification neural network that is pre-trained for classification of objects.

In aspect 10, the method of any of aspects 7-9 further includes that the neural network is configured with a classification algorithm based on at least one of a random forest algorithm, a multilayer perceptron (MLP) neural network algorithm, a logistic regression algorithm, a naive Bayes machine learning algorithm, or a support vector machine (SVM) algorithm.

In aspect 11, the method of any of aspects 7-10 further includes that the input includes both the imaging information and the clinical information associated with the aneurysm patient and the method including generating the outcome prediction for the aneurysm treatment based on both the imaging information and the clinical information.

In aspect 12, the method of any of aspects 7-11 further includes that the clinical information includes at least one of: demographic information for the aneurysm patient, aneurysm information associated with the imaging information, dimension information for an aneurysm imaged in the imaging information, allergies of the aneurysm patient, medication information for the aneurysm patient, or pre-existing condition information for the aneurysm patient.

In aspect 13, the method of any of aspects 7-11 further includes that the outcome prediction comprises at least one of: one or more measurements for an aneurysm imaged in the imaging information, or a best predicted size of an intrasaccular device for the aneurysm imaged in the imaging information.

In aspect 14, the method of aspect 13 further includes that the imaging information includes at least one annotation identifying a region of the aneurysm.

In aspect 15, the method of aspect 13 further includes that the imaging information includes raw imaging information, and the method further includes sending, to the user device, an identification of a presence of the aneurysm in the imaging information in addition to the one or more measurements.

In aspect 16, the method of aspect 15 further includes that the identification includes a contour outlining an aneurysm sac imaged in the imaging information.

In aspect 17, the method of any of aspects 7-16 further includes sending, to the user device, an identification of at least one treatment device for implant in an aneurysm sac of the aneurysm patient based on the at least one of the imaging information or the clinical information for the aneurysm patient, the at least one treatment device identified based on having a highest predicted likelihood of complete occlusion of the aneurysm sac imaged in the imaging information for the aneurysm patient from a set of potential treatment devices.

In aspect 18, the method of aspect 17 further includes that the identification includes a list of multiple treatment devices and a respective outcome prediction associated with each treatment device in the list of multiple treatment devices.

In aspect 19, the method of aspect 18 further includes that the multiple treatment devices include different sizes of a same type of intrasaccular implant device having a most favorable outcome prediction from the set of potential treatment devices.

In aspect 20, the method of aspect 18 further includes that the multiple treatment devices include different types of aneurysm treatment devices.

In aspect 21, the method of any of aspects 7-20 further includes that the outcome prediction comprises a size for an intrasaccular device having a highest likelihood of complete occlusion.

In aspect 22, the method of any of aspects 7-21 further includes the imaging information comprises one or more of: magnetic resonance imaging (MRI) information, magnetic resonance angiography (MRA) information, a computed tomography (CT) scan information, a two-dimensional (2D) digital subtraction angiography information, or a three-dimensional (3D) reconstruction from a sequence of 2D images.

In aspect 23, the method of any of aspects 7-22 further includes the outcome prediction indicates a predicted likelihood of the complete occlusion of an aneurysm sac imaged in the imaging information.

Aspect 24 is an apparatus for providing outcome predictions for intrasaccular implant devices based on imaging and clinical information, the apparatus including memory; and at least one processor coupled to the memory, and based at least in part on information stored in the memory, configured to the method of any of aspects 7-23.

Aspect 25 is an apparatus for providing outcome predictions for intrasaccular implant devices based on imaging and clinical information, the apparatus including means for performing the method of any of aspects 7-23.

Aspect 26 is a non-transitory computer-readable storage medium storing computer executable code for providing outcome predictions for intrasaccular implant devices based on imaging and clinical information, the code when executed by a processor causes the processor to perform the method of any of aspects 7-23.

Aspect 27 is a method for providing segmentation information obtained for an aneurysm sac at neural network apparatus, the method including: receiving digital imaging information for a patient; segmenting the aneurysm sac within the digital imaging information using a trained neural network model with an encoder comprising an image classification neural network that is pre-trained for classification of objects; and outputting segmentation information for the aneurysm sac.

In aspect 28, the method of aspect 27 further includes that the image classification neural network is pre-trained for the classification of objects different than aneurysms.

In aspect 29, the method of aspect 27 or aspect 28 further includes that the trained neural network model further comprises a decoder trained to segment aneurysm sacs in images and configured to output measurement information for the aneurysm sac after the digital imaging information is processed at the encoder.

In aspect 30, the method of any of aspects 27-29 further include that the digital imaging information comprises one or more two-dimensional (2D) digital subtraction angiography (DSA) images of a wide-neck bifurcation aneurysm prior to implantation of an intrasaccular device.

In aspect 31, the method of any of aspects 27-30 further include that the digital imaging information includes one or more of a lateral and anterior-posterior (AP) views on a two-dimensional (2D) digital subtraction angiography (DSA) image or a three-dimensional (3D) axial slice stack reconstructed from a sequence of DSA images.

In aspect 32, the method of any of aspects 27-31 further include that the digital imaging information comprises an annotation or adjustment identifying the aneurysm sac, and the at least one processor is configured to semi-automatically segment the aneurysm sac using the trained neural network model.

In aspect 33, the method of any of aspects 27-32 further include that the digital imaging information comprises a raw angiography image, and the method further including identifying a presence of the aneurysm sac in the raw angiography image using the trained neural network model prior to automatic segmentation of the aneurysm sac.

In aspect 34, the method of any of aspects 27-33 further includes outputting a recommended size of an intrasaccular device for implant at the aneurysm sac.

In aspect 35, the method of any of aspects 27-34 further includes indicating a likelihood of full occlusion for the aneurysm sac.

Aspect 36 is an apparatus for providing segmentation information obtained for an aneurysm sac, the apparatus including memory; and at least one processor coupled to the memory, and based at least in part on information stored in the memory, configured to the method of any of aspects 27-35.

Aspect 37 is an apparatus for providing segmentation information obtained for an aneurysm sac, the apparatus including means for performing the method of any of aspects 27-35.

Aspect 38 is a non-transitory computer-readable storage medium storing computer executable code for providing segmentation information obtained for an aneurysm sac, the code when executed by a processor causes the processor to perform the method of any of aspects 27-35.

Aspect 39 is a method for obtaining outcome predictions for intrasaccular implant devices based on digital imaging information and clinical information for an aneurysm patient, the method including: providing, to a neural network module a request associated with at least one of digital imaging information or clinical information for an aneurysm patient; and receiving, in a response from the neural network module, at least one of an outcome prediction for the aneurysm treatment, a first identification of an aneurysm in the imaging information, one or more measurements for the aneurysm, or a second identification of at least one treatment device for implant in an aneurysm sac.

In aspect 40, the method of aspect 39 further includes displaying, at a user interface, the outcome prediction, the first identification, the second identification, or the one or more measurements.

In aspect 41, the method of aspect 39 or 40 further includes that the neural network component is remote from the user device and the request is provided to the neural network module and the response is received from the neural network module via a communication interface at the user device.

In aspect 42, the method of aspect 39 or 40 further includes that the neural network component is comprised in the user device.

Aspect 43 is an apparatus for providing segmentation information obtained for an aneurysm sac, the apparatus including memory; and at least one processor coupled to the memory, and based at least in part on information stored in the memory, configured to the method of any of aspects 39-42.

Aspect 44 is an apparatus for providing segmentation information obtained for an aneurysm sac, the apparatus including means for performing the method of any of aspects 39-42.

Aspect 45 is a non-transitory computer-readable storage medium storing computer executable code for providing segmentation information obtained for an aneurysm sac, the code when executed by a processor causes the processor to perform the method of any of aspects 39-42.

What is claimed is:

1. A neural network apparatus for providing outcome predictions for intrasaccular implant devices based on digital imaging and clinical information, the apparatus comprising:
memory; and
at least one processor coupled to the memory, and based at least in part on information stored in the memory, configured to:
receive, as input from a user device, digital imaging information and the clinical information for an aneurysm patient;
generate, using a neural network trained for aneurysm outcome prediction, the digital imaging information, and the clinical information, an outcome prediction for at least one intrasaccular implant device for implant in an aneurysm sac identified in the digital imaging information and having a highest predicted likelihood of complete occlusion of the aneurysm sac from a set of potential treatment devices; and
output, for display on a device, an identification of the at least one intrasaccular implant device and the outcome prediction for each of the at least one intrasaccular implant device.

2. The neural network apparatus of claim 1, wherein the neural network is configured with a classification algorithm based on at least one of a random forest algorithm, a multilayer perceptron (MLP) neural network algorithm, a logistic regression algorithm, a naive Bayes machine learning algorithm, or a support vector machine (SVM) algorithm.

3. The neural network apparatus of claim 1, wherein, based at least in part on the information stored in the memory, the at least one processor is further configured to perform at least one of:
semi-automatic segmentation of the digital imaging information to obtain one or more measurements of the aneurysm sac by passing the digital imaging information through an encoder to obtain code and through a decoder to output the one or more measurements of the aneurysm sac based on the code, or
automatic segmentation of raw imaging information to identify the aneurysm sac and to obtain the one or more measurements of the aneurysm sac by passing the digital imaging information through the encoder to obtain the code and through the decoder to output the one or more measurements of the aneurysm sac based on the code,
wherein the outcome prediction for each of the at least one intrasaccular implant device is based on the one or more measurements obtained for the aneurysm sac, dimensions of the at least one intrasaccular implant device, and the clinical information for the aneurysm patient.

4. A system for providing outcome predictions for intrasaccular implant devices based on imaging and clinical information, the system comprising:
memory; and
at least one processor coupled to the memory, and based at least in part on information stored in the memory, configured to:
receive, as input from a user device, at least one of imaging information or the clinical information associated with an aneurysm patient;
generate an outcome prediction for an aneurysm treatment of the aneurysm patient based on the at least one of the imaging information or the clinical information received as the input; and
send, to a display, the outcome prediction for the aneurysm treatment for display at the user device.

5. The system of claim 4, wherein the system includes a neural network trained for aneurysm outcome prediction, and the at least one processor is configured to process the imaging information and the clinical information using the neural network to generate the outcome prediction for the aneurysm treatment.

6. The system of claim 5, wherein the neural network includes an encoder and decoder, the encoder comprising an image classification neural network that is pre-trained for classification of objects.

7. The system of claim 6, wherein the neural network is configured with a classification algorithm based on at least one of a random forest algorithm, a multilayer perceptron (MLP) neural network algorithm, a logistic regression algorithm, a naive Bayes machine learning algorithm, or a support vector machine (SVM) algorithm.

8. The system of claim 4, wherein the input includes both the imaging information and the clinical information associated with the aneurysm patient and the at least one processor is configured to generate the outcome prediction for the aneurysm treatment based on both the imaging information and the clinical information.

9. The system of claim 4, wherein the clinical information includes at least one of:
demographic information for the aneurysm patient,
aneurysm information associated with the imaging information,
dimension information for an aneurysm imaged in the imaging information,
allergies of the aneurysm patient,
medication information for the aneurysm patient, or
pre-existing condition information for the aneurysm patient.

10. The system of claim 4, wherein the outcome prediction comprises at least one of:
one or more measurements for an aneurysm imaged in the imaging information, or
a best predicted size of an intrasaccular device for the aneurysm imaged in the imaging information.

11. The system of claim 10, wherein the imaging information includes at least one annotation identifying a region of the aneurysm.

12. The system of claim 11, wherein the imaging information includes raw imaging information, and based at least in part on the information stored in the memory, the at least one processor is further configured to:
send, to the user device, an identification of a presence of the aneurysm in the imaging information in addition to the one or more measurements.

13. The system of claim 12, wherein the identification includes a contour outlining an aneurysm sac imaged in the imaging information.

14. The system of claim 4, wherein, based at least in part on the information stored in the memory, the at least one processor is further configured to:
send, to the user device, an identification of at least one treatment device for implant in an aneurysm sac of the aneurysm patient based on the at least one of the imaging information or the clinical information for the aneurysm patient, the at least one treatment device identified based on having a highest predicted likelihood of complete occlusion of the aneurysm sac imaged in the imaging information for the aneurysm patient from a set of potential treatment devices.

15. The system of claim 14, wherein the identification includes a list of multiple treatment devices and a respective outcome prediction associated with each treatment device in the list of multiple treatment devices.

16. The system of claim 15, wherein the multiple treatment devices include different sizes of a same type of intrasaccular implant device having a most favorable outcome prediction from the set of potential treatment devices.

17. The system of claim 15, wherein the multiple treatment devices include different types of aneurysm treatment devices.

18. The system of claim 15, wherein the outcome prediction comprises a size for an intrasaccular device having a highest likelihood of complete occlusion.

19. The system of claim 4, wherein the imaging information comprises one or more of:
magnetic resonance imaging (MRI) information,
magnetic resonance angiography (MRA) information,
a computed tomography (CT) scan information,
a two-dimensional (2D) digital subtraction angiography information, or
a three-dimensional (3D) reconstruction from a sequence of 2D images.

20. The system of claim 4, wherein the outcome prediction indicates a predicted likelihood of a complete occlusion of an aneurysm sac imaged in the imaging information.

21. A neural network apparatus for providing segmentation information obtained for an aneurysm sac, the apparatus comprising:
memory; and
at least one processor coupled to the memory, and based at least in part on information stored in the memory, configured to:
receive digital imaging information for a patient;
segment the aneurysm sac within the digital imaging information using a trained neural network model with an encoder comprising an image classification neural network that is pre-trained for classification of objects; and
output segmentation information for the aneurysm sac.

22. The neural network apparatus of claim 21, wherein the image classification neural network is pre-trained for the classification of objects different than aneurysms.

23. The neural network apparatus of claim 21, wherein the trained neural network model further comprises a decoder trained to segment aneurysm sacs in images and configured to output measurement information for the aneurysm sac after the digital imaging information is processed at the encoder.

24. The neural network apparatus of claim 21, wherein the digital imaging information comprises one or more two-dimensional (2D) digital subtraction angiography (DSA) images of a wide-neck bifurcation aneurysm prior to implantation of an intrasaccular device.

25. The neural network apparatus of claim 21, wherein the digital imaging information includes one or more of a lateral and anterior-posterior (AP) views on a two-dimensional (2D) digital subtraction angiography (DSA) image or a three-dimensional (3D) axial slice stack reconstructed from a sequence of DSA images.

26. The neural network apparatus of claim 21, wherein the digital imaging information comprises an annotation or adjustment identifying the aneurysm sac, and the at least one processor is configured to semi-automatically segment the aneurysm sac using the trained neural network model.

27. The neural network apparatus of claim 21, wherein the digital imaging information comprises a raw angiography image, and the at least one processor is further configured to:
identify a presence of the aneurysm sac in the raw angiography image using the trained neural network model prior to automatic segmentation of the aneurysm sac.

28. The neural network apparatus of claim 21, wherein the at least one processor is further configured to:
output a recommended size of an intrasaccular device for implant at the aneurysm sac.

29. The neural network apparatus of claim 21, wherein the at least one processor is further configured to:
indicate a likelihood of full occlusion for the aneurysm sac.
